(19) United States
(12) Patent Application Publication
(10) Pub. No.: US 2015/0306209 A1
(43) Pub. Date: Oct. 29, 2015

(54) CANINE PARVOVIRUS TYPE 2c ISOLATES AND METHODS OF USE

(71) Applicant: INTERVET INC., Summit, NJ (US)

(72) Inventors: Terri L. Wasmoen, Omaha, NE (US); Nallakannu P. Lakshmanan, Millsboro, DE (US); Kenneth A. Stachura, Omaha, NE (US); Jessica A. Moore, Georgetown, DE (US); Thomas Clinton Gore, Salisbury, MD (US)

(21) Appl. No.: 14/651,744

(22) PCT Filed: Dec. 18, 2013
(86) PCT No.: PCT/EP2013/077023

§ 371 Date: Jun. 12, 2015

Related U.S. Application Data
(60) Provisional application No. 61/739,067, filed on Dec. 19, 2012.
(60) Provisional application No. 61/778,751, filed on Mar. 13, 2013.

(51) Int. Cl. A61K 39/23; C12N 7/00

(52) U.S. Cl. CPC A61K 39/23; C12N 7/00; C12N 2750/14321; C12N 2750/14334

(57) ABSTRACT

The present invention discloses an attenuated canine parvovirus. In addition, the present invention discloses isolated and/or recombinant canine parvovirus capsid proteins and the nucleic acids that encode the canine parvovirus capsid proteins. The present invention further discloses immunogenic compositions and/or vaccines comprising the attenuated canine parvovirus isolates, corresponding capsid proteins, and/or recombinant vectors which express nucleic acids that encode the canine parvovirus capsid proteins.

### FIELD OF THE INVENTION

The present invention relates to attenuated type 2c canine parvoviruses (CPV-2c). In addition, the present invention relates to isolated and/or recombinant CPV-2c capsid proteins and to the nucleic acids that encode these CPV-2c capsid proteins. The present invention further relates to immunogenic compositions and/or vaccines comprising the attenuated CPV-2c isolates, corresponding capsid proteins, and/or recombinant vectors which express nucleic acids that encode the canine parvovirus capsid proteins.

### BACKGROUND

Canine parvovirus (CPV) is primarily an enteric pathogen that infects dogs, particularly young dogs, and is characterized by acute diarrhea, fever, and leukopenia in dogs and puppies more than 4 to 5 weeks old. Even very young puppies can suffer myocardial disease. The mortality rate from canine parvovirus (CPV) is relatively high and vaccines that protect puppies/dogs from canine parvovirus are among the most common and important canine vaccines.

CPV is a single-stranded DNA virus that has a genome of about 5200 bases within a lone nucleic acid segment [Parrish and Kawaoka, Annu Rev. Microbiol., 59:553-586 (2005)]. This DNA segment contains two open reading frames, each of which encode at least two proteins due to alternative mRNA splicing. The two components of the virus capsid, VP1 and VP2, are encoded by one of the open reading frames, whereas the other open reading frame encodes two nonstructural proteins: NS1 and NS2. VP2 is the major immunogenic CPV capsid protein. The primary host binding partner for VP2 is the transferrin receptor [Hueffer et al., J. Virol. 77:1718-1726 (2003)]. NS1 has been identified as a helicase and is essential for genome replication and protein production [Niskanen et al., J. of Virol. 84(10):5391-5403 (2010)].

CPV was first isolated in 1978 and was named CPV-2 to distinguish it from canine parvovirus Minute virus (CMV or CPV-1). Approximately a year after the initial isolation of CPV-2, a genetic variant, CPV-2a, was identified. In the mid-1980's, a second genetic variant, CPV-2b, was identified. CPV-2a and CPV-2b soon completely displaced CPV-2. Today, CPV-2a is no longer detected in the United States [Parrish and Kawaoka, Annu Rev. Microbiol., 59:553-586 (2005)]. A fourth CPV variant in this family, CPV-2c, was first described in 2000 and has been reported in Italy [Buonavoglia et al., J. Gen. Virol. 82:3021-3025 (2001)], in Vietnam [Nakamura et al., Clin. Diag. Lab. Immuno. 8(3) 663-668 (2001)] and in Spain [Nakamura et al., Arch. Virol., 149:2261-2269 (2004); Decaro et al., J. Vet. Med. B Infect. Dis. Vet. Public Health, 53(10):468-72 (2006)] and more recently in the United States [U.S. Pat. No. 8,227,593; U.S. Pat. No. 8,258,274; Hong et al., J. Vet. Diagn. Invest. (5):535-9 (2007)].

The amino acid sequence of the major capsid protein (VP2) of CPV changed relatively little as CPV progressed from CPV-2 to CPV-2a, to CPV-2b, and to CPV-2c. The amino acid changes in the VP2 protein between CPV-2 to CPV-2a are: a methionine residue (M₈₇) at position 87 to a leucine residue (L₈₇), an isoleucine residue (I₁₀₁) to a threonine (T₁₀₁), an alanine residue at position 300 (A₃₀₀) to a glycine residue (G₃₀₀), an aspartic acid at 305 (D₃₀₅) to a tyrosine residue (Y₃₀₅) and a valine residue at position 555 (V₅₅₅) to an isoleucine residue (I₅₅₅) [see e.g., Stucker et al., J. Virol. 86(3):1514-1521 (2012)], although more recently strains of CPV-2a have been identified with apparent reversions of the valine residue at position 555 back to the isoleucine residue. The VP2 proteins of CPV-2a and CPV-2b differ at two amino acid residue positions: the asparagine residue at position 426 (N₄₂₆) of CPV-2a was replaced with an aspartic acid residue (D₄₂₆) in CPV-2b, and the isoleucine residue at position 555 (I₅₅₅) in CPV-2a was replaced with a valine residue (V₅₅₅) in CPV-2b. As indicated above, the I₅₅₅ to V₅₅₅ change is actually a reversion back to the original CPV type 2 amino acid sequence. The VP2 protein of CPV-2c differs from that of CPV-2b by only a single amino acid difference: the aspartic acid residue at position 426 (D₄₂₆) is replaced by a glutamic acid residue (E₄₂₆) [see, Spibey et al., Veterinary Microbiology 128:48-55 (2007); U.S. Pat. No. 8,227,583 B2; U.S. Pat. No. 8,258,274 B2].

Indeed, changes at position 426 of VP2 from N₄₂₆ of CPV-2 and CPV-2a, to D₄₂₆ of CPV-2b, and then to E₄₂₆ of CPV-2c appear to have significantly altered the antigenic structure of the corresponding viruses [Parrish and Kawaoka, Annu Rev. Microbiol., 59:553-586 (2005)]. More recently, CPV-2c isolates have been identified that have an additional amino acid change in their VP2 protein: the threonine residue at position 440 (T₄₄₀) has been converted to an alanine residue (A₄₄₀) in these isolates [U.S. Pat. No. 8,227,583 B2; U.S. Pat. No. 8,258,274 B2]. This variant has been reported to cause disease in vaccinated animals, and accordingly, it has been suggested to include this variant in future canine parvovirus vaccines [U.S. Pat. No. 8,227,583 B2; U.S. Pat. No. 8,258,274 B2].

CPV is most closely related to feline parvovirus (FPV) and is generally regarded as a genetic variant of FPV. FPV, which is also known as feline panleukopenia virus, is the etiological cause of feline panleukopenia, a highly contagious disease that is common in unvaccinated kittens. FPV infections cause leukopenia, fever, diarrhea, and often can be fatal. CPV is also genetically and antigenically related to the parvoviruses that infect minks, foxes, raccoons, and other carnivores.

FPV and CPV isolates have complex host ranges. For example, FPV isolates can infect canines, whereas the original CPV-2 isolates could not replicate in cats, though subsequent variants of CPV-2, i.e., CPV-2a, CPV-2b, and CPV-2c, can. On the other hand, both FPV and CPV isolates can readily infect feline cells, but only CPV isolates infect canine cells [Parrish and Kawaoka, Annu Rev. Microbiol., 59:553-586 (2005)]. In addition, the pH dependence of hemagglutination of CPV isolates differ appreciably from that of FPV isolates, i.e., whereas FPV isolates only hemagglutinate below pH 6.8, CPV isolates hemagglutinate over the pH range of 6.0 to 8.0 [Chang et al., J. Virol. 66(12) 6858-6867 (1992)].

Insight into the role of a number of amino acid residues in the VP2 protein of FPV and CPV has been obtained through recombination mapping and mutagenesis [Chang et al., J. Virol. 66(12) 6858-6867 (1992); Parrish and Kawaoka, Annu Rev. Microbiol., 59:553-586 (2005)]. For example, Parrish and Kawaoka [Annul. Rev. Microbiol., 59:553-586 (2005)] reported that amino acid substitutions of both a lysine residue at position 93 (K₉₃) by an asparagine residue (N₉₃) and an aspartic acid residue at position 323 (D₃₂₃) by an asparagine residue (N₃₂₃) enabled FPV to bind the host cell canine transferrin receptor and infect canine cells, although neither substitution alone was capable of introducing either property. Moreover, it has been reported that either replacing a glycine residue at position 299 (G₂₉₉) with a glutamic acid residue (E₂₉₉) or replacing an alanine residue at position 300 (A₃₀₀) with an aspartic acid residue (D₃₀₀) prevents canine parvovirus both from binding the host cell canine transferrin receptor and from infecting the cells or dogs [Parrish and Kawaoka, Annu Rev. Microbiol., 59:553-586 (2005)]. Amino acid positions: 80, 564, and 568 of the VP2 protein also have been reported to influence host range.

In addition, following repeated passages in Norden Laboratories feline kidney (NLFK) cells, a nonhemagglutinating mutant of a 1978 isolate of canine parvovirus was obtained and found to comprise a VP2 protein having a lysine residue at position 377 (K₃₇₇) in place of the native arginine residue (R₃₇₇). This change was reported to eliminate all virus binding to erythrocytes [Parrish et al., Virology 163(1) 230-232 (1988); Chang et al., J. Virol. 66(12) 6858-6867 (1992)]. More recently, it has been shown that changing an isoleucine residue at position 219 (I₂₁₉) to a valine residue (V₂₁₉) and a glutamine residue at position 386 (Q₃₈₆) to a lysine residue (K₃₈₆) of a CPV-2c VP2 protein enhanced the attenuation of a recombinant canine parvovirus that comprises a heterogenous CPV-2c/CPV-2 genome, i.e., the region encoding the capsid proteins is from a CPV-2c isolate and the region encoding the nonstructural proteins is from a CPV-2 isolate [WO2011107534 (A1); WO2012007589 (A1)].

In the U.S. all canine parvovirus vaccines currently are directed against only the CPV-2, CPV-2a and/or CPV-2b variants. Although a vaccine comprising a live attenuated CPV-2 isolate (NOBIVAC® DHPPi) was shown to protect dogs against a CPV-2c challenge [Spibey et al., Veterinary Microbiology 128:48-55 (2007)], it is generally believed that vaccines containing a CPV-2c VP2 antigen would be desireable. Indeed, unlike CPV-2, CPV-2a, and CPV-2b that primarily infect puppies, CPV-2c appears to have a greater affinity for infecting adult dogs [U.S. Pat. No. 8,227,593; U.S. Pat. No. 8,258,274]. Therefore, there is a need to develop new canine parvovirus vaccines comprising a CPV-2c VP2 antigen in order to increase the certainty of providing protection to canines against CPV-2c. Moreover, because multivalent vaccines are often preferable to monovalent vaccines, there also is a need to develop new multivalent vaccines comprising a CPV-2c and/or a CPV-2c VP2 antigen.

The citation of any reference herein should not be construed as an admission that such reference is available as “prior art” to the instant application.

### SUMMARY OF THE INVENTION

Accordingly, the present invention provides novel attenuated canine parvovirus type 2c (CPV-2c) isolates. In addition, the present invention provides isolated and/or recombinant polypeptides from CPV-2c isolates, including the capsid protein. Moreover, the present invention provides nucleic acids that encode the CPV-2c polypeptides and recombinant vectors that comprise and express such nucleic acids. The present invention further provides immunogenic compositions comprising the attenuated canine parvovirus isolates, corresponding polypeptides, e.g., capsid proteins, and/or recombinant vectors which express nucleic acids that encode the CPV-2c polypeptides. Furthermore, the present invention provides vaccines, including multivalent vaccines, that comprise the attenuated CPV-2c isolates, and/or corresponding polypeptides, and/or recombinant vectors which express nucleic acids that encode the CPV-2c polypeptides.

In one aspect the present invention provides an isolated attenuated canine parvovirus type 2c (CPV-2c) isolate that comprises a genome that encodes a capsid protein comprising an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃), and/or 219 (K₂₁₉), and/or 377 (K₃₇₇). In related embodiments, the isolated attenuated CPV-2c isolate comprises a genome that encodes a capsid protein comprising an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆) and a serine residue at position 300 (S₃₀₀), and/or an alanine residue at position 301 (A₃₀₁), and/or an isoleucine residue at position 555 (I₅₅₅). In still other embodiments the isolated attenuated CPV-2c isolate comprises a genome that encodes a capsid protein comprising an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃), and/or 219 (K₂₁₉), and/or 377 (K₃₇₇) and further comprises a serine residue at position 300 (S₃₀₀), and/or an alanine residue at position 301 (A₃₀₁), and/or an isoleucine residue at position 555 (I₅₅₅).

In more particular embodiments, the isolated attenuated CPV-2c isolate comprises a genome that encodes a capsid protein comprising an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃), and/or 219 (K₂₁₉), and/or 377 (K₃₇₇) and further comprises a serine residue at position 300 (S₃₀₀). In other particular embodiments the isolated attenuated CPV-2c isolate comprises a genome that encodes a capsid protein comprising an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃), and/or 219 (K₂₁₉), and/or 377 (K₃₇₇), and further comprises an alanine residue at position 301 (A₃₀₁). In still other particular embodiments the isolated attenuated CPV-2c isolate comprises a genome that encodes a capsid protein comprising an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃), and/or 219 (K₂₁₉), and/or 377 (K₃₇₇), and further comprises an isoleucine residue at position 555 (I₅₅₅).

In even more particular embodiments, the isolated attenuated CPV-2c isolate comprises a genome that encodes a capsid protein comprising an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃), and 219 (K₂₁₉). In related embodiments of this type, the isolated attenuated CPV-2c isolate comprises a genome that encodes a capsid protein comprising an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃) and 377 (K₃₇₇). In still other related embodiments of this type, the isolated attenuated CPV-2c isolate comprises a genome that encodes a capsid protein comprising an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 219 (K₂₁₉) and 377 (K₃₇₇). In other embodiments the isolated attenuated CPV-2c isolate comprises a genome that encodes a capsid protein comprising an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃), and 219 (K₂₁₉), and 377 (K₃₇₇).

In a more particular embodiment the isolated attenuated CPV-2c isolate comprises a genome that encodes a capsid protein comprising the amino acid sequence of SEQ ID NO: 2. In another embodiment of this type, the isolated attenuated CPV-2c isolate comprises a genome that encodes capsid proteins comprising the amino acid sequences of SEQ ID NO: 2 and SEQ ID NO: 4, respectively. In a related embodiment, the isolated attenuated CPV-2c isolate comprises a genome that encodes a capsid protein comprising the amino acid sequence of SEQ ID NO: 2 and a nonstructural protein comprising the amino acid sequence of SEQ ID NO: 6. In yet another embodiment, the isolated attenuated CPV-2c isolate comprises a genome that encodes a capsid protein comprising the amino acid sequence of SEQ ID NO: 2 and a nonstructural protein comprising the amino acid sequence of SEQ ID NO: 8. In still another embodiment, the isolated attenuated CPV-2c isolate comprises a genome that encodes a capsid protein comprising the amino acid sequence of SEQ ID NO: 2, a capsid protein comprising the amino acid sequence of SEQ ID NO: 4, and a nonstructural protein comprising the amino acid sequence of SEQ ID NO: 6. In yet another embodiment, the isolated attenuated CPV-2c isolate comprises a genome that encodes a capsid protein comprising the amino acid sequence of SEQ ID NO: 2, a capsid protein comprising the amino acid sequence of SEQ ID NO: 4, and a nonstructural protein comprising the amino acid sequence of SEQ ID NO: 8. In still another embodiment, the isolated attenuated CPV-2c isolate comprises a genome that encodes a capsid protein comprising the amino acid sequence of SEQ ID NO: 2, a capsid protein comprising the amino acid sequence of SEQ ID NO: 4, a nonstructural protein comprising the amino acid sequence of SEQ ID NO: 6, and a nonstructural protein comprising the amino acid sequence of SEQ ID NO: 8.

The present invention also provides an isolated attenuated CPV-2c isolate that comprises a genome comprising an open reading frame that comprises nucleotides 2286 to 4541 of SEQ ID NO: 9. The present invention further provides an isolated attenuated CPV-2c isolate that comprises a genome comprising an open reading frame comprises nucleotides 273 to 2279 of SEQ ID NO: 9. In a related embodiment, an isolated attenuated CPV-2c isolate comprises a genome comprising open reading frames that comprise nucleotides 273 to 2279 and nucleotides 2286 to 4541 of SEQ ID NO: 9. In a particular embodiment of this type, the genome comprises the nucleotide sequence of SEQ ID NO. 9. In still a more specific embodiment of this type, the isolated attenuated CPV-2c isolate has the ATCC accession No. PTA-13492. In a related embodiment the isolated attenuated CPV-2c isolate comprises all of the identifying characteristics of ATCC accession No. PTA-13492.

The present invention further provides immunogenic compositions and vaccines. In particular embodiments, a vaccine or immunogenic composition of the present invention comprises an isolated attenuated CPV-2c isolate of the present invention. In addition a vaccine or immunogenic composition of the present invention can be a multivalent vaccine (or multivalent immunogenic composition). In more specific embodiments, a multivalent vaccine of the present invention combines a CPV-2c isolate of the present invention (either live attenuated or killed) with one or more live attenuated or killed canine and/or feline antigens. In certain embodiments a CPV-2c isolate of the present invention is combined with a canine distemper virus. In other embodiments a CPV-2c isolate of the present invention is combined with a canine adenovirus type 2. In yet other embodiments a CPV-2c isolate of the present invention is combined with a canine parvovirus type 2b. In still other embodiments a CPV-2c isolate of the present invention is combined with a canine parainfluenza virus. In yet other embodiments a CPV-2c isolate of the present invention is combined with a canine coronavirus. In still other embodiments a CPV-2c isolate of the present invention is combined with a canine pneumovirus. In yet other embodiments a CPV-2c isolate of the present invention is combined with an infectious canine hepatitis virus. In still other embodiments a CPV-2c isolate of the present invention is combined with a canine herpes virus. In yet other embodiments a CPV-2c isolate of the present invention is combined with a rabies virus. In still other embodiments a CPV-2c isolate of the present invention is combined with a canine minute virus. In yet other embodiments a CPV-2c isolate of the present invention is combined with a canine influenza virus. In alternative embodiments a CPV-2c isolate of the present invention is combined with a pseudorabies virus. In other alternative embodiments, a CPV-2c isolate of the present invention is combined with a live attenuated Bordetella bronchiseptica. In related embodiments, a CPV-2c isolate of the present invention is combined with a Bordetella bronchiseptica bacterin.

Multivalent vaccines of the present invention can further include three or more canine antigens, e.g., a CPV-2c isolate of the present invention combined with a canine adenovirus type 2 and a canine distemper virus, or a CPV-2c isolate of the present invention combined with a canine parainfluenza virus and a canine distemper virus. In still other embodiments, the multivalent vaccines of the present invention can further include four or more canine antigens, e.g., a CPV-2c isolate of the present invention combined with a canine parainfluenza virus, a canine distemper virus and canine adenovirus type 2. Similarly, a live attenuated or killed CPV-2c isolate of the present invention can be combined with live, killed or recombinant canine antigens.

In a particular embodiment, a live attenuated CPV-2c isolate of the present invention is combined with a live attenuated canine parainfluenza virus, a live attenuated canine distemper virus, and a live attenuated canine adenovirus type 2. In another such embodiment, the multivalent vaccine of the present invention includes a live attenuated CPV-2c isolate of the present invention combined with a live attenuated canine parainfluenza virus, a live attenuated canine distemper virus, a live attenuated canine adenovirus type 2, and a live attenuated canine coronavirus.

In addition, a live attenuated (or killed) CPV-2c isolate of the present invention can be combined in a multivalent vaccine with one or more live attenuated or killed feline antigens including one or more of the following antigens: feline herpesvirus (FHV), feline calicivirus antigen (FCV), feline parvovirus (FPV), feline leukemia virus (FeLV), feline infectious peritonitis virus (FIPV), feline immunodeficiency virus (FIV), borna disease virus (BDV), rabies virus, feline influenza virus, feline pneumovirus, Chlamydophila felis, Bordetella bronchiseptica, and Bartonella spp. (e.g., B. henselae).

In particular embodiments, a vaccine of the present invention can comprise a pharmaceutically acceptable carrier. The present invention further provides methods of immunizing a canine or feline against CPV comprising administering a vaccine (e.g., a multivalent vaccine) of the present invention to the canine or feline. In a particular embodiment of this type, a vaccine of the present invention is administered to a canine by parenteral administration. In a more particular embodiment of this type, the administering is performed subcutaneously.

The present invention further provides isolated and/or recombinant CPV-2c proteins, including chimeric proteins (e.g., a fusion protein), isolated and/or recombinant nucleic acids that encode such proteins and chimeric proteins, and recombinant vectors that comprise these recombinant nucleic acids and which can express the proteins and/or chimeric proteins of the present invention. In particular embodiments the present invention provides a capsid protein comprising an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃), and/or 219 (K₂₁₉), and/or 377 (K₃₇₇). In related embodiments, the capsid protein comprises an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆) and a serine residue at position 300 (S₃₀₀), and/or an alanine residue at position 301 (A₃₀₁), and/or an isoleucine residue at position 555 (I₅₅₅). In still other embodiments the capsid protein comprises an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃), and/or 219 (K₂₁₉), and/or 377 (K₃₇₇) and further comprises a serine residue at position 300 (S₃₀₀), and/or an alanine residue at position 301 (A₃₀₁), and/or an isoleucine residue at position 555 (I₅₅₅).

In more particular embodiments, the capsid protein comprises an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃), and/or 219 (K₂₁₉), and/or 377 (K₃₇₇) and further comprises a serine residue at position 300 (S₃₀₀). In other particular embodiments the capsid protein comprises an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃), and/or 219 (K₂₁₉), and/or 377 (K₃₇₇), and further comprises an alanine residue at position 301 (A₃₀₁). In still other particular embodiments the capsid protein comprises an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃), and/or 219 (K₂₁₉), and/or 377 (K₃₇₇), and further comprises an isoleucine residue at position 555 (I₅₅₅).

In even more particular embodiments, the capsid protein comprises an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃), and 219 (K₂₁₉). In related embodiments of this type, the capsid protein comprises an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃) and 377 (K₃₇₇). In still other related embodiments of this type, the capsid protein comprises an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 219 (K₂₁₉) and 377 (K₃₇₇). In other embodiments the capsid protein comprises an amino acid sequence that comprises 95%, or 98%, or 99% or greater identity with the amino acid sequence of SEQ ID NO: 2; wherein the amino acid sequence of the capsid protein comprises a glutamic acid residue at position 426 (E₄₂₆), and a lysine residue at amino acid positions 93 (K₉₃), and 219 (K₂₁₉), and 377 (K₃₇₇).

In a more particular embodiment the capsid protein comprises the amino acid sequence of SEQ ID NO: 2. In another embodiment the capsid protein comprises the amino acid sequence of SEQ ID NO: 4. In a related embodiment, a nonstructural protein comprises the amino acid sequence of SEQ ID NO: 6. In yet another embodiment, a nonstructural protein comprises the amino acid sequence of SEQ ID NO: 8.

The present invention also provides isolated, recombinant, or both isolated and recombinant nucleic acids that can encode any of the CPV-2c proteins of the present invention. In a particular embodiment the nucleic acid comprises the nucleotide sequence of SEQ ID NO: 1. In another embodiment the nucleic acid comprises the nucleotide sequence of SEQ ID NO: 3. In yet another embodiment the nucleic acid comprises the nucleotide sequence of SEQ ID NO: 5. In still another embodiment the nucleic acid comprises the nucleotide sequence of SE ID NO: 7. In yet another embodiment the nucleic acid comprises the nucleotide sequence of nucleotides 2286 to 4541 of SEQ ID NO: 9. In still another embodiment the nucleic acid comprises the nucleotide sequence of nucleotides 273 to 2279 of SEQ ID NO: 9. In a particular embodiment of this type, the nucleic acid comprises the nucleotide sequence of SEQ ID NO. 9.

The present invention also provides recombinant vectors that comprise the nucleic acids of the present invention. In particular embodiments the recombinant vectors are recombinant expression vectors. In certain embodiments of this type, the recombinant expression vector is a recombinant viral vector.

The CPV-2c proteins and corresponding recombinant vectors can be included together with, or alternatively, in place of the CPV-2c isolates of the present invention, in any of the immunogenic compositions or vaccines of the present invention. Thus, any of the multivalent vaccines of the present invention can comprise a recombinant expression vector encoding and expressing a CPV-2c capsid protein, e.g., the VP2 protein, and/or antigenic fragments of the CPV-2c VP2 protein of the present invention in place of and/or together with a live attenuated CPV-2c isolate of the present invention.

These and other aspects of the present invention will be better appreciated by reference to the following Figures and the Detailed Description.

### BRIEF DESCRIPTION OF THE FIGURES

FIG. 1 shows the serum cross neutralization (SN) of two test CPV-2c isolates, isolate #4, ATCC accession No. PTA-13492, or isolate #12, with a standard CPV-2 isolate and assorted CPV-2c isolates. All of the CPV-2c isolates, including the two test isolates have a threonine residue at amino acid position 440 (T₄₄₀) of their VP2 capsid protein. This is consistent with the sequence found for all VP2 proteins of CPV-2c isolates that were initially identified.

FIG. 2 shows the serum cross neutralization (SN) of two test CPV-2c isolates, isolate #4, ATCC accession No. PTA-13492, or isolate #12, with a standard CPV-2 isolate and assorted CPV-2c Isolates. All of the CPV-2c isolates, other than the two test isolates, have an alanine residue at amino acid position 440 (A₄₄₀) of their VP2 capsid protein. A VP2 protein having a substitution of an alanine residue for the threonine residue at position 440 has been reported for a significant number of CPV-2c isolates in recent years.

### DETAILED DESCRIPTION OF THE INVENTION

While the amino acid sequence of the major capsid protein of canine parvovirus, VP2, has changed relatively modestly over the 35 years since the discovery of this virus, the antigenicity of CPV has been significantly altered several times over this period, with each change resulting in the later variant completely displacing the earlier variant as the disease causing agent. Should this paradigm continue, the most prevalent CPV in the U.S. may soon become CPV-2c. Therefore, it is only prudent to replace and/or supplement existing CPV vaccines that had been designed to protect against the earlier variants with a vaccine designed to protect against CPV-2c. Towards this end, the present invention provides vaccines, including multivalent vaccines, that comprise live attenuated and/or killed CPV-2c isolates and/or CPV-2c VP2 antigens and/or recombinant vectors encoding the CPV-2c VP2 antigens.

Therefore in one aspect, the present invention provides novel attenuated canine parvovirus type 2c isolates that comprise a genome that encodes a VP2 capsid protein comprising a glutamic acid residue at position 426 (E₄₂₆) and a lysine residue at position 93 (K₉₃) in place of an asparagine residue, a lysine residue at position 219 (K₂₁₉) in place of an isoleucine residue, and a lysine residue at position 377 (K₃₇₇) in place of an arginine residue, relative to typical wild type CPV-2c strains. Surprisingly, despite the fact that two of these amino acid changes involve the substitution of a neutral amino acid with a positively charged amino acid residue, thereby causing a change in the overall charge of the VP2 protein, this isolate was found to have superior serum neutralization properties when ascertained with either a standard CPV-2 isolate or a variety of CPV-2c field isolates, including those having the recently reported A₄₄₀ modification [U.S. Pat. No. 8,227,583 B2; U.S. Pat. No. 8,258,274 B2]. More surprisingly, the presence of a lysine residue at position 93 (K₉₃) of the VP2 protein had adversely affected the binding of FPV isolates to the canine transferrin receptor, and the presence of a lysine residue at position 377 (K₃₇₇) of its VP2 protein had eliminated the ability of an earlier CPV variant to bind erythrocytes (see, Table 1 below). Neither of these attributes would, a priori, be desireable in a new live vaccine strain. However, as a constituent of a live multivalent vaccine, this CPV-2c isolate was found to be fully attenuated in canines, and furthermore, protected vaccinated puppies against a CPV-2b challenge.

The amino acid sequence of the VP2 capsid protein of the particular isolate described in the Examples below (ATCC accession No. PTA-13492) actually has six (6) amino acid residue modifications relative to that of the corresponding prevalent VP2 amino acid sequence for CPV-2c. Aside from comprising lysine residues at position 93 (K₉₃), at position 219 (K₂₁₉), and at position 377 (K₃₇₇), the amino acid sequence also comprises an isoleucine residue at position 555 (I₅₅₅) in place of a valine residue, a serine residue at position 300 (S₃₀₀) in place of a glycine residue, and an alanine residue at position 301 (A₃₀₁) in place of a threonine residue (SEQ ID NO: 2). All six of these modifications appear to be unique for a CPV-2c isolate, i.e. unique/identifying characteristics of ATCC accession No. PTA-13492, though as noted above, at least five of the six sites had been noted earlier in one or more of the earlier CPV variants, or FPV.

Therefore, in a particular aspect of the present invention, attenuated CPV-2c isolates (attenuated or killed) are provided which share the unique/identifying characteristics of the canine parvovirus ATCC accession No. PTA-13492. In another aspect of the present invention, an isolated and/or recombinant capsid protein obtained from such isolates are provided. Included in the present invention are novel antigenic fragments of the capsid proteins of the invention. In a related aspect, isolated and/or recombinant nucleic acids encoding the capsid proteins and/or encoding antigenic fragments of the capsid proteins are provided. In a further aspect, the present invention provides recombinant vectors, including recombinant virus vectors that comprise and/or express such nucleic acids.

The present invention further provides vaccines against canine parvovirus comprising any of these isolates (live and/or killed), and/or isolated and/or recombinant capsid proteins, and/or novel antigenic fragments of the capsid proteins, and/or recombinant nucleic acids encoding the capsid proteins and/or encoding antigenic fragments of the capsid proteins (including recombinant viruses that comprise and/or express such nucleic acids), either individually or in any combination. The vaccines and immunogenic compositions of the present invention can be administered to the subject animal (e.g., canine) by any method. In particular embodiments a vaccine of the present invention is administered by injection through the parenteral route, e.g., subcutaneously. In other embodiments a vaccine of the present invention is administered by oral administration.

In addition, the present invention provides related booster vaccines which can be administered by the same way as the primary vaccine, or by an alternative method.

As used herein the following terms will have the following meaning:

As used herein the term, “canine” includes all domestic dogs, Canis lupus familiaris or Canis familiaris, unless otherwise indicated.

As used herein, the term “feline” refers to any member of the Felidae family. Members of this family include wild, zoo, and domestic members, such as any member of the subfamilies Felinae, Panterinae or Acinonychinae. Nonlimiting examples of species included within the Felidae family are cats, lions, tigers, pumas, jaguars, leopards, snow leopards, panthers, North American mountain lions, cheetahs, lynx, bobcats, caracals or any cross breeds thereof. Cats also include domestic cats, pure-bred and/or mongrel companion cats, show cats, laboratory cats, cloned cats and wild or feral cats.

As used herein, the terms “protecting” or “providing protection to” and “aids in the protection” do not require complete protection from any indication of infection. For example, “aids in the protection” can mean that the protection is sufficient such that, after challenge, symptoms of the underlying infection are at least reduced, and/or that one or more of the underlying cellular, physiological, or biochemical causes or mechanisms causing the symptoms are reduced and/or eliminated. It is understood that “reduced,” as used in this context, means relative to the state of the infection, including the molecular state of the infection, not just the physiological state of the infection.

As used herein, a multivalent vaccine is a vaccine that comprises two or more different antigens. In a particular embodiment of this type, the multivalent vaccine stimulates the immune system of the recipient against two or more different pathogens.

As used herein, the term “pharmaceutically acceptable” is used adjectivally to mean that the modified noun is appropriate for use in a pharmaceutical product. When it is used, for example, to describe an excipient in a pharmaceutical vaccine, it characterizes the excipient as being compatible with the other ingredients of the composition and not disadvantageously deleterious to the intended recipient animal, e.g., canine.

“Parenteral administration” includes subcutaneous injections, submucosal injections, intravenous injections, intramuscular injections, intradermal injections, and infusion.

As used herein the term “polypeptide” is used interchangeably with the term “protein” and is further meant to encompass peptides. Therefore, as used herein, a polypeptide is a polymer of two or more amino acids joined together by peptide linkages. Preferably, a polypeptide is a polymer comprising twenty or more amino acid residues joined together by peptide linkages, whereas a peptide comprises two to twenty amino acid residues joined together by peptide linkages.

As used herein a polypeptide “consisting essentially of” or that “consists essentially of” a specified amino acid sequence is a polypeptide that (i) retains an important characteristic of the polypeptide comprising that amino acid sequence, e.g., the antigenicity of at least one epitope of the inventive capsid protein(s), and (ii) further comprises the identical amino acid sequence(s), except it consists of plus or minus 10% (or a lower percentage), and preferably plus or minus 5% (or a lower percentage) of the amino acid residues. In a particular embodiment, additional amino acid residues included as part of the polypeptide are part of a linked Tag, such as a C-terminal His₆ Tag. In the specific case of the CPV-2c VP2 protein of the present invention, the polypeptide (i) retains the antigenicity of at least one epitope of the inventive capsid protein(s), and (ii) further comprises the identical amino acid sequence(s), except it consists of plus or minus 5% (or a lower percentage) of the amino acid residues, yet still retains a glutamic acid residue at position 426 (E₄₂₆) and at least one, preferably at least two, more preferably at least three, and most preferably all six of the unique amino acid residues as defined in Table 1 below, i.e., a lysine residue at position 93 (K₉₃), a lysine residue at position 219 (K₂₁₉), a lysine residue at position 377 (K₃₇₇), an isoleucine residue at position 555 (I₅₅₅), a serine residue at position 300 (S₃₀₀), and/or an alanine residue at position 301 (A₃₀₁), as defined by the amino acid sequence of SEQ ID NO: 2.

A molecule is “antigenic” when it is capable of specifically interacting with an antigen recognition molecule of the immune system, such as an immunoglobulin (antibody) or T cell antigen receptor. An antigenic polypeptide (and/or fragment of the polypeptide) contains at least 6, and preferably at least 12 or more amino acid residues. An antigenic portion of a molecule can be that portion that is immunodominant for recognition by an antibody or a T cell receptor, and/or it can be a portion used to generate an antibody to the molecule by conjugating an immunogenic portion of the antigen to a carrier molecule for immunization. A molecule that is antigenic need not be itself immunogenic, i.e., capable of eliciting an immune response without a carrier.

As used herein the term “antigenic fragment” of a particular protein is a fragment of that protein that is antigenic. For example, an antigenic fragment of a CPV-2c capsid protein of the present invention can be any antigenic fragment that retains a glutamic acid residue at position 426 (E₄₂₆) and at least one, preferably at least two, more preferably at least three, and most preferably all six of the unique amino acid residues as defined in Table 1 below, i.e., a lysine residue at position 93 (K₉₃), a lysine residue at position 219 (K₂₁₉), a lysine residue at position 377 (K₃₇₇), an isoleucine residue at position 555 (I₅₅₅), a serine residue at position 300 (S₃₀₀), and/or an alanine residue at position 301 (A₃₀₁), as defined by the amino acid sequence of SEQ ID NO: 2, including large fragments that retains are missing as little as a single amino acid from the full-length protein. In a particular embodiment, an antigenic fragment of a CPV-2c capsid protein of the present invention contains between 60 and 580 amino acid residues. In yet another embodiment, an antigenic fragment contains 100 amino acid residues or more, but fewer than 500 amino acid residues. In still another embodiment, an antigenic fragment contains 250 amino acid residues or more, but fewer than 500 amino acid residues. In yet another embodiment, an antigenic fragment contains 300 amino acid residues or more, but fewer than 500 amino acid residues.

An antigenic fragment of a CPV-2c capsid protein of the present invention can be obtained from a recombinant source, from a protein isolated from natural sources, or through chemical synthesis. Similarly, an antigenic fragment can be obtained following the proteolytic digestion of such CPV-2c capsid proteins or fragments thereof. Alternatively, an antigenic fragment of the present invention can be generated by recombinant expression, or alternatively, through peptide synthesis.

As used herein the term “chimeric protein” is used interchangeably with the terms “chimeric polypeptide” and “chimeric peptide” and is meant to include fusion proteins, polypeptides, and peptides. A “chimeric protein” comprising a CPV-2c capsid protein of the present invention comprises at least a portion of the CPV-2c capsid protein that retains a glutamic acid residue at position 426 (E₄₂₆) and at least one, preferably at least two, more preferably at least three, and most preferably all six of the unique amino acid residues as defined in Table 1 below, i.e., a lysine residue at position 93 (K₉₃), a lysine residue at position 219 (K₂₁₉), a lysine residue at position 377 (K₃₇₇), an isoleucine residue at position 555 (I₅₅₅), a serine residue at position 300 (S₃₀₀), and/or an alanine residue at position 301 (A₃₀₁), as defined by the amino acid sequence of SEQ ID NO: 2 joined via a peptide bond to at least a portion of a different protein. A chimeric protein of the present invention also can comprise two or more different proteins and/or portions thereof. Chimeric proteins of the present invention also can have additional structural, regulatory, and/or catalytic properties. As used herein a chimeric protein can contain multiple additions to at least a portion of a given protein, e.g., a chimeric protein can comprise both a His₆Tag and an epitope from another antigen. In a particular embodiment, the non-capsid portion of the chimeric protein functions as a means of detecting and/or isolating the chimeric protein or fragment thereof after a recombinant nucleotide encoding the given protein or antigenic fragment thereof is expressed. Non-CPV-2c capsid protein amino acid sequences are generally, but not always, either amino- or carboxy-terminal to the protein sequence.

As used herein one amino acid sequence is 100% “identical” to a second amino acid sequence when the amino acid residues of both sequences are identical. Accordingly, an amino acid sequence is 50% “identical” to a second amino acid sequence when 50% of the amino acid residues of the two amino acid sequences are identical. The sequence comparison is performed over a contiguous block of amino acid residues comprised by a given protein, e.g., a protein, or a portion of the polypeptide being compared. In a particular embodiment, selected deletions or insertions that could otherwise alter the correspondence between the two amino acid sequences are taken into account.

As used herein, nucleotide and amino acid sequence percent identity can be determined using C, MacVector (MacVector, Inc. Cary, N.C. 27519), Vector NTI (Informax, Inc. MD), Oxford Molecular Group PLC (1996) and the Clustal W algorithm with the alignment default parameters, and default parameters for identity. These commercially available programs can also be used to determine sequence similarity using the same or analogous default parameters. Alternatively, an Advanced Blast search under the default filter conditions can be used, e.g., using the GCG (Genetics Computer Group, Program Manual for the GCG Package, Version 7, Madison, Wis.) pileup program using the default parameters.

As used herein a “nucleic acid” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. When referring to a nucleic acid that is double stranded both the “sense” strand and the complementary “antisense” strand are intended to be included. Thus a nucleic acid that is hybridizable to SEQ ID NOs: 1, for example, can be either hybridizable to the “sense” strand of the respective sequence, or to the “antisense” strand which can be readily determined from the respective sense strands listed in the Sequence Listing provided herein. The individual components of a nucleic acid are referred to as nucleotides.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A coding sequence is “under the control” of transcriptional and translational control sequences in a cell when RNA polymerase transcribes the coding sequence into mRNA, which can then be trans-RNA spliced, if, when, and where appropriate, and translated into the protein encoded by the coding sequence.

As used herein, the term “encodes” in the context of a CPV-2c isolate comprising a genome encoding a protein comprising a given amino acid sequence not only includes proteins that have coding sequences that are uninterrupted in the genome such as the VP2 capsid protein (e.g., encoded by nucleotides 2787-4541 of SEQ ID NO: 9) and the NS1 protein (e.g., encoded by nucleotides 273-2279 of SEQ ID NO: 9), but can also include those proteins that are encoded through alternative mRNA splicing such as VP1 (e.g., encoded by nucleotides 2286-2315 . . . 2388-4541 of SEQ ID NO: 9) and NS2 (e.g., encoded by nucleotides 273-533 . . . 2006-2242 of SEQ ID NO: 9).

A nucleotide sequence is “operatively linked” to an expression control sequence when the expression control sequence controls or regulates the transcription and translation of that nucleotide sequence. The term operatively linked includes having an appropriate start signal.

A “heterologous nucleotide sequence” as used herein is a nucleotide sequence that is added by recombinant methods to a nucleotide sequence encoding a polypeptide of the present invention or encoding a fragment thereof (La, an antigenic fragment), to form a nucleic acid that is not naturally formed in nature. Such nucleic acids can e.g., encode chimeric proteins. In addition, as used herein, a heterologous nucleotide sequence need not be a single contiguous nucleotide sequence, but can include multiple non-contiguous nucleotide sequences that have been combined with a nucleotide sequence encoding a polypeptide of the present invention, or a portion thereof. A heterologous nucleotide sequence can comprise non-coding sequences including restriction sites, regulatory sites, promoters and the like. In still another embodiment the heterologous nucleotide can function as a means of detecting a nucleic acid of the present invention.

### Preparation of Attenuated and/or Killed CPV-2c

Live attenuated vaccines may be prepared by the conventional means as detailed in the Example 1 below. Conventional means generally include, for example, modifying pathogenic strains by in vitro passaging, cold adaptation, modifying the pathogenicity of the organism by genetic manipulation, preparation of chimeras, insertion of antigens into viral vectors, selecting non-virulent wild type strains, and other methods well known to the skilled artisan.

In some embodiments, the live attenuated CPV-2c strain is derived by serial passage of the wild-type virus through cell culture. In alternative embodiments, an attenuated strain is derived by serial passage of the wild-type virus through laboratory animals, non-host animals, or eggs. The accumulation of genetic mutation during such passage(s) typically leads to progressive loss of virulence of the organism to the original host.

In some embodiments, the live attenuated virus strain is prepared by co-infection of permissible cells with an attenuated mutant virus and pathogenic virus. The desired resultant recombinant virus has the safety of the attenuated virus with genes coding for protective antigens from the pathogenic virus.

In some embodiments, the live attenuated virus strain is prepared by cold adaptation. A cold-adapted virus has an advantage of replicating only at the temperature found in upper respiratory tract. A method of generation of a cold-adapted equine influenza virus has been described in U.S. Pat. No. 6,177,082 [hereby incorporated by reference in its entirety]. A desired resulting cold-adapted virus confers one or more of the following phenotypes: cold adaptation, temperature sensitivity, dominant interference, and/or attenuation.

In some embodiments, the live attenuated virus strain is prepared by recombinant means, such as by recombinant recombination, a point mutation, deletion, or insertion to convert a pathogenic virus to a non-pathogenic or less-pathogenic virus compared to the original virus, while preserving the protective properties of the original virus. In some embodiments, the live attenuated virus is prepared by cloning the candidate of genes of protective antigens into a genome of a non-pathogenic or less-pathogenic canine parvovirus, or other virus or organism.

Alternatively, inactivation of a CPV-2c isolate of the present invention can be accomplished by treating the virus with inactivation chemicals [e.g., formalin, beta propiolactone (“BPL”), bromoethylamine (“BEA”), and binary ethylenimine (“BEI”)] or by non-chemical methods [e.g., heat, freeze/thaw, or sonication] to disable or decrease the replication capacity of the virus.

### Vaccines and Multivalent Vaccines

The vaccines of the present invention can comprise any of the CPV-2c isolates of the present invention (live and/or killed), and/or corresponding isolated and/or recombinant capsid proteins, and/or novel antigenic fragments of the capsid proteins, and/or recombinant nucleic acids encoding the capsid proteins and/or encoding antigenic fragments of the capsid proteins (including recombinant vectors, such as recombinant viruses, that comprise and express such nucleic acids), either individually or in any combination.

In addition, any of such CPV-2c antigens can be included in a multivalent vaccine. Such multivalent vaccines can comprise live or killed antigens of and/or from other canine or feline pathogens including subunit antigens and/or corresponding recombinant vectors that expressing such subunit antigens from other canine and/or feline pathogens. For example, a multivalent vaccine could include a CPV-2c antigen of the present invention along with a recombinant myxoma virus expressing a feline and/or canine influenza virus hemagglutinin.

In particular embodiments a multivalent vaccine comprises an isolated CPV-2c isolate of the present invention that further comprises a canine canine distemper virus, and/or a canine adenovirus type 2, and/or a canine parvovirus type 2b, and/or a canine parainfluenza virus, and/or a canine coronavirus, and/or a canine influenza virus, and/or a canine pneumovirus. These viruses can be live attenuated or alternatively killed viruses.

The vaccines, including multivalent vaccines, of the present invention may include one or more excipients that enhance an animal subject's immune response (which may include an antibody response, cellular response, or both), thereby increasing the effectiveness of the vaccine. Use of such excipients (or “adjuvants”) may be particularly beneficial when using an inactivated vaccine. The adjuvant(s) may be a substance that has a direct (e.g., cytokine or Bacille Calmette-Guerin (“BCG”)) or indirect effect (liposomes) on cells of the canine patients immune system. Examples of often suitable adjuvants include oils (e.g., mineral oils) water and oil adjuvants, metallic salts (e.g., aluminum hydroxide, such as Alhydrogel, or aluminum phosphate), bacterial components (e.g., bacterial liposaccharides, Freund's adjuvants, and/or MDP), plant components (e.g., Quil A), and/or one or more substances that have a carrier effect (e.g., bentonite, latex particles, liposomes, and/or Quil A, ISCOM), or combination of these. As noted above, adjuvants also include, for example, CARBIGEN™ adjuvant and acrylic block copolymers such as CARBOPOL. It should be recognized that the present invention encompasses both vaccines that comprise an adjuvant(s), as well as vaccines that do not comprise any adjuvant.

It is also contemplated that the vaccine may be freeze-dried (or otherwise reduced in liquid volume) for storage, and then reconstituted in a liquid before or at the time of administration. Such reconstitution may be achieved using, for example, vaccine-grade water. In certain embodiments, a vaccine of the present invention can be formed into freeze-dried compositions, such as spheres, e.g., as produced by a method previously described [see e.g., WO 2010/125084; US 2012/0049412 A1, hereby incorporated by reference in their entireties].

Stabilizer components may also be included in the vaccines. Appropriate stabilizers include: sugars and sugar alcohols (such as sucrose, dextrose, trehalose, sorbitol) and gelatin protein hydrolysates (lactalbumin hydrolysate, NZ Amine) serum albumin (bovine serum albumin, ovalbumin) and buffering compounds.

### Vaccine Administration

It is contemplated that a vaccine of the present invention may be administered to the animal subject, e.g., a canine, a single time; or, alternatively, two or more times over days, weeks, months, or years. In some embodiments, the vaccine is administered at least two times. In some such embodiments, for example, the vaccine is administered twice, with the second dose (e.g., a booster) being administered at least about 2 weeks after the first. In some embodiments, the vaccine is administered twice, with the second dose being administered no greater than 8 weeks after the first. In some embodiments, the second dose is administered at from about 2 weeks to about 4 years after the first dose, from about 2 to about 8 weeks after the first dose, or from about 3 to about 4 weeks after the first dose. In some embodiments, the second dose is administered about 4 weeks after the first dose. The first and subsequent dosages may vary, such as, for example, in amount and/or form. Often, however, the dosages are the same as to amount and form. When only a single dose is administered, the amount of vaccine in that dose alone generally comprises a therapeutically effective amount of the vaccine. When, however, more than one dose is administered, the amounts of vaccine in those doses together may constitute a therapeutically effective amount.

The preferred composition of the vaccine depends on, for example, whether the vaccine is an inactivated vaccine, live attenuated vaccine, or both. It also depends on the method of administration of the vaccine. It is contemplated that the vaccine may comprise one or more conventional pharmaceutically acceptable carriers, adjuvants, other immune-response enhancers, and/or vehicles (collectively referred to as “excipients”). Such excipients are generally selected to be compatible with the active ingredient(s) in the vaccine. Use of excipients is generally known to those skilled in the art.

The vaccines may be administered by conventional means, including, for example, parenteral administration (such as, without limitation, subcutaneous or intramuscular administration) or mucosal administration, (such as intranasal, oral, intratracheal, and ocular). The vaccines may also be administered (including, without limitation, via a skin patch, scarification, or topical administration). As seen in Example 2, the subcutaneous injection of a multivalent vaccine comprising a live attenuated CPV-2c isolate of the present invention proved to successfully protect against a virulent CPV-2b challenge.

It is also contemplated that the vaccine may be administered via the animal subject's drinking water and/or food. It is further contemplated that the vaccine may be administered in the form of a treat, toy, or by supralingual administration [see e.g., WO 2011/008958].

The vaccines (including multivalent vaccines) of the present invention may be administered as part of a combination therapy, i.e., a therapy that includes, in addition to the vaccine itself, administering one or more additional active agents, adjuvants, therapies, etc. In that instance, it should be recognized the amount of vaccine that constitutes a “therapeutically effective” amount may be more or less than the amount of vaccine that would constitute a “therapeutically effective” amount if the vaccine were to be administered alone. Other therapies may include those known in the art, such as, for example, anti-viral medications, analgesics, fever-reducing medications, expectorants, anti-inflammation medications, antihistamines, antibiotics to treat bacterial infection that result as a secondary infection to a canine parvovirus infection, and/or administration of fluids.

### Nucleic Acids Encoding the CPV-2c Capsid Proteins of the Present Invention

A nucleic acid, such as a cDNA, that encodes a CPV-2c capsid protein, e.g., VP2 protein of the present invention, can be placed into a vector, e.g., a recombinant bacterial host cell, to express a protein and/or antigen of the present invention. Alternatively, the vector can be a recombinant virus (e.g., a rabbit myxoma virus) to be used in immunogenic compositions such as vaccines.

In addition, obtaining and/or constructing a DNA that encodes a CPV-2c capsid protein of the present invention, including antigenic fragments thereof, facilitates the production of economically important quantities of the protein or antigenic fragments thereof. The large quantities of the proteins and/or antigenic fragments thereof produced are useful for making certain vaccines of the present invention.

Accordingly, the present invention provides nucleotide constructs that allow for the expression and isolation of large quantities of the proteins and/or antigens of the present invention, such as the CPV-2c capsid protein. These nucleic acids can further contain heterologous nucleotide sequences. To express a recombinant protein of the present invention in a host cell, an expression vector can be constructed comprising the corresponding cDNA. The present invention therefore, provides expression vectors containing nucleic acids encoding the CPV-2c capsid proteins of the present invention, including variants thereof, and/or antigenic fragments thereof and/or chimeric proteins.

Due to the degeneracy of nucleotide coding sequences, other DNA sequences which encode substantially the same amino acid sequence as a nucleic acid encoding a CPV-2c capsid protein of the present invention may be used in the practice of the present invention. These include, but are not limited to, allelic genes, homologous genes from other strains, and/or those that are altered by the substitution of different codons that encode the same amino acid residue within the sequence, thus producing a silent change. Host cells comprising the expression vectors of the present invention are also provided. One particular host cell is an E. coli cell.

General methods for the cloning of cDNAs and expression of their corresponding recombinant proteins have been described [see Sambrook and Russell, Molecular Cloning, A laboratory Manual, 3^{rd} edition, Cold Spring Harbor Laboratory Press, Cold Spring Harbor L.I. (2000)]. Preferably, all of the nucleic acid constructs of the present invention are sequence confirmed.

In addition, any technique for mutagenesis known in the art can be used to modify a CPV-2c capsid protein of the present invention, including but not limited to, in vitro site-directed mutagenesis [Hutchinson et al., J. Biol. Chem., 253:6551 (1978); Zoller and Smith, DNA, 3:479-488 (1984); Oliphant et al., Gene, 44:177 (1986); Hutchinson et al., Proc. Natl. Acad. Sci. U.S.A., 83:710 (1986); Wang and Malcolm, BioTechniques 26:680-682 (1999) the contents of which are hereby incorporated by reference in their entireties]. The use of TAB@ linkers (Pharmacia), etc. and PCR techniques also can be employed for site directed mutagenesis [see Higuchi, “Using PCR to Engineer DNA”, in PCR Technology: Principles and Applications for DNA Amplification, H. Erlich, ed., Stockton Press, Chapter 6, pp. 61-70 (1989)].

The present invention also provides nucleic acids that hybridize to nucleic acids comprising the nucleotide sequences of the present invention. A nucleic acid is “hybridizable” to another nucleic acid, such as a cDNA, genomic DNA, or RNA, when a single stranded form of the nucleic acid can anneal to the other nucleic acid under the appropriate conditions of temperature and solution ionic strength [see Sambrook and Russell, Molecular Cloning, A laboratory Manual, 3^{rd} edition, Cold Spring Harbor Laboratory Press, Cold Spring Harbor L.I. (2000)].

The conditions of temperature and ionic strength determine the “stringency” of the hybridization. For preliminary screening for homologous nucleotides, low stringency hybridization conditions, corresponding to a Tₘ of 55° C., can be used, e.g., 5× saline sodium citrate (SSC), 0.1% sodium dodecyl sulfate (SDS), 0.25% milk, and no formamide; or 30% formamide, 5×SSC, 0.5% SDS. Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art.

The greater the degree of similarity or homology between two nucleotide sequences, the greater the value of Tₘ for hybrids of nucleotides having those sequences. The relative stability (corresponding to higher Tₘ) of nucleotide hybridizations decreases in the following order: RNA:RNA, DNA:RNA, DNA:DNA. For hybrids of greater than 100 nucleotides in length, equations for calculating Tₘ have been derived [see Sambrook and Russell, Molecular Cloning, A laboratory Manual, 3^{rd} edition, Cold Spring Harbor Laboratory Press, Cold Spring Harbor L.I. (2000)]. For hybridization with shorter nucleic acids, i.e., oligonucleotides, the position of mismatches becomes more important, and the length of the oligonucleotide determines its specificity.

Depending upon circumstances a suitable minimal length for a hybridizable nucleic acid can be at least about 12 nucleotides; or at least about 18 nucleotides; or the length can be at least about 24 nucleotides; or at least about 36 nucleotides. Alternatively, the minimum length can be at least about 48 or at least about 72 nucleotides, or longer, as indicated above. In certain embodiments the nucleic acid is between 12 and 72 nucleotides long. In other embodiments the nucleic acid is between 18 and 48 nucleotides long. In yet other embodiment the nucleic acid is between 1800 and 2010 nucleotides long. In still other embodiments the nucleic acid is between 1200 to 2010 nucleotides long.

In a specific embodiment, the term “standard hybridization conditions” refers to a Tₘ of 55° C., and utilizes conditions as set forth above. Under more stringent conditions, the Tₘ is 60° C., and under even more stringent conditions, the Tₘ is 65° C. for both hybridization and wash conditions, respectively.

### Recombinant Vectors

The present invention also provides vectors that comprise the nucleic acids and express the proteins of the present invention. Such vectors can contain one or more nucleotide sequences and/or heterologous sequences of the present invention operatively linked to an expression control sequence. In certain embodiments the vector is an animal virus vector. Examples of such vectors include adenoviruses, herpesviruses, poxviruses, paramyxoviruses, rhabdoviruses, and baculoviruses. In other embodiments, the vector is a plasmid or a bacterium such as E. coli. Any of the vectors of the present invention can be used in a vaccine.

### CPV-2c Proteins of the Present Invention

The present invention provides isolated and/or recombinant CPV-2c capsid proteins, including antigen fragments and chimeric proteins thereof. In addition, CPV-2c capsid proteins containing altered sequences in which functionally equivalent amino acid residues are substituted for those within the amino acid sequence resulting in a conservative amino acid substitution are also provided by the present invention.

Thus, one or more of these amino acid residues within the sequence can be substituted by another amino acid of a similar polarity, which can, but not necessarily, act as a functional equivalent, resulting in a silent alteration. Substitutes for an amino acid within the sequence may be selected from other members of the class to which the amino acid belongs. For example, the nonpolar amino acids include alanine, leucine, isoleucine, valine, proline, phenylalanine, tryptophan and methionine. The polar neutral amino acids include glycine, serine, threonine, cysteine, tyrosine, asparagine, and glutamine.

The positively charged (basic) amino acids include arginine and lysine. The negatively charged (acidic) amino acids include aspartic acid and glutamic acid.

Particularly preferred conserved amino acid exchanges are:

(a) Lys for Arg or vice versa such that a positive charge may be maintained;
(b) Glu for Asp or vice versa such that a negative charge may be maintained;
(c) Ser for Thr or vice versa such that a free —OH can be maintained;
(d) Gln for Asn or vice versa such that a free NH₂ can be maintained; and
(e) Ile for Leu or for Val or vice versa as being roughly equivalent hydrophobic amino acids.

All of the CPV-2c capsid proteins of the present invention, including antigenic fragments thereof, also can be part of a chimeric protein. In a specific embodiment, a chimeric polypeptide is expressed in a prokaryotic cell. Such a chimeric protein can be a fusion protein used to isolate a CPV-2c capsid protein of the present invention, through the use of an affinity column that is specific for a protein fused to the CPV-2c capsid protein, for example. Examples of such fusion proteins include: a glutathione-S-transferase (GST) fusion protein, a maltose-binding protein (MBP) fusion protein, a FLAG-tagged fusion protein, or a poly-histidine-tagged fusion protein. Specific linker sequences such as a Ser-Gly linker can also be part of such a fusion protein.

Indeed, the expression of one or more of the inventive proteins, as a fusion protein, can facilitate stable expression, and/or allow for purification based on the properties of the fusion partner. Thus the purification of the recombinant CPV-2c capsid proteins of the present invention can be simplified through the use of fusion proteins having affinity Tags. For example, GST binds glutathione conjugated to a solid support matrix, MBP binds to a maltose matrix, and poly-histidine chelates to a Ni-chelation support matrix [see Hochuli et al., Biotechnology 6:1321-1325 (1998)].

The fusion protein can be eluted from the specific matrix with appropriate buffers, or by treating with a protease that is specific for a cleavage site that has been genetically engineered in between the CPV-2c capsid protein, for example, and its fusion partner. Alternatively, a CPV-2c capsid protein can be combined with a marker protein such as green fluorescent protein [Waldo et al., Nature Biotech. 17:691-695 (1999); U.S. Pat. No. 5,625,048 and WO 97/26333, the contents of which are hereby incorporated by reference in their entireties].

Alternatively or in addition, other column chromatography steps (e.g., gel filtration, ion exchange, affinity chromatography etc.) can be used to purify the recombinant polypeptides of the present invention (see below). In many cases, such column chromatography steps employ high performance liquid chromatography or analogous methods in place of the more classical gravity-based procedures.

In addition, a CPV-2c capsid protein of the present invention or an antigenic fragment thereof can be chemically synthesized [see e.g., Synthetic Peptides: A User's Guide, W.H. Freeman & Co., New York, N.Y., pp. 382, Grant, ed. (1992)].

### General Polypeptide Purification Procedures

Generally, initial steps for purifying a polypeptide of the present invention can include salting in or salting out, in ammonium sulfate fractionations; solvent exclusion fractionations, e.g., an ethanol precipitation; detergent extractions to free membrane bound polypeptides, using such detergents as TRITON X-100, TWEEN-20 etc.; or high salt extractions. Solubilization of membrane proteins may also be achieved using aprotic solvents such as dimethyl sulfoxide and hexamethylphosphoramide. In addition, high speed ultracentrifugation may be used either alone or in conjunction with other extraction techniques.

Generally good secondary isolation or purification steps include solid phase absorption using calcium phosphate gel, hydroxyapatite, or solid phase binding. Solid phase binding may be performed through ionic bonding, with either an anion exchanger, such as diethylaminoethyl (DEAE), or diethyl[2-hydroxypropyll aminoethyl (QAE) SEPHADEX or cellulose; or with a cation exchanger such as carboxymethyl (CM) or sulfopropyl (SP) SEPHADEX or cellulose. Alternative means of solid phase binding includes the exploitation of hydrophobic interactions e.g., the use of a solid support such as phenylSepharose and a high salt buffer; affinity-binding immuno-binding, using e.g., a inventive protein bound to a suitable anti-CPV-2c capsid protein selective antibody bound to an activated support. Other solid phase supports include those that contain specific dyes or lectins etc.

A further solid phase support technique that is often used at the end of the purification procedure relies on size exclusion, such as SEPHADEX and SEPHAROSE gels. Alternatively, a pressurized or centrifugal membrane technique, using size exclusion membrane filters may be employed. Oftentimes, these two methodologies are used in tandem.

Solid phase support separations are generally performed batch-wise with low-speed centrifugation, or by column chromatography. High performance liquid chromatography (HPLC), including such related techniques as FPLC, is presently the most common means of performing liquid chromatography. Size exclusion techniques may also be accomplished with the aid of low speed centrifugation. In addition size permeation techniques such as gel electrophoretic techniques may be employed. These techniques are generally performed in tubes, slabs or by capillary electrophoresis.

Almost all steps involving polypeptide purification employ a buffered solution. Unless otherwise specified, generally 25-100 mM concentrations of buffer salts are used. Low concentration buffers generally imply 5-25 mM concentrations. High concentration buffers generally imply concentrations of the buffering agent of between 0.1-2.0 M concentrations. Typical buffers can be purchased from most biochemical catalogues and include the classical buffers such as Tris, pyrophosphate, monophosphate and diphosphate and the Good buffers such as Mes, Hepes, Mops, Tricine and Ches [Good et al., Biochemistry, 5:467 (1966); Good and Izawa, Meth. Enzymol., 24B:53 (1972); and Fergunson and Good, Anal. Biochem., 104:300 (1980].

Materials to perform all of these techniques are available from a variety of commercial sources such as Sigma Chemical Company in St. Louis, Mo.

### Antibodies to the CPV-2c Capsid Proteins of the Present Invention

The CPV-2c capsid proteins of the present invention, and antigenic fragments thereof, as produced by a recombinant source, or through chemical synthesis, or as isolated from natural sources; and variants, derivatives or analogs thereof, including fusion proteins, may be used as an immunogen to generate antibodies. Such antibodies include but are not limited to polyclonal, monoclonal, chimeric including single chain, Fab fragments, and a Fab expression library. Such antibodies can be used in diagnostic kits or as components in vaccines.

Specific anti-CPV-2c capsid protein antibodies of the invention, for example, may be cross-reactive, that is, they may recognize closely-related CPV-2c capsid proteins obtained from a different source (e.g., a different CPV-2c isolate). Polyclonal antibodies have greater likelihood of cross-reactivity. Alternatively, an antibody of the invention may be specific for a single form of an inventive protein, for example, such as a specific fragment of the capsid protein that has the amino acid sequence of SEQ ID NO: 2, or a closely related variant thereof.

In a particular aspect of the present invention compositions and uses of antibodies that are immunoreactive with only a CPV-2c capsid protein of the present invention are provided. Such antibodies “bind specifically” to the particular CPV-2c capsid protein protein respectively, meaning that they bind via antigen-binding sites of the antibody as compared to non-specific binding interactions.

The terms “antibody” and “antibodies” are used herein in their broadest sense, and include, without limitation, intact monoclonal and polyclonal antibodies as well as fragments such as Fv, Fab, and F(ab′) fragments, single-chain antibodies such as scFv, and various chain combinations. The antibodies may be prepared using a variety of well-known methods including, without limitation, immunization of animals having native or transgenic immune repertoires, phage display, hybridoma and recombinant cell culture.

Both polyclonal and monoclonal antibodies may be prepared by conventional techniques. [See, for example, Monoclonal Antibodies, Hybridomas: A New Dimension in Biological Analyses, Kennet et al. (eds.), Plenum Press, New York 37 (1980); and Antibodies: A Laboratory Manual, Harlow and Land (eds.), Cold Spring Harbor Laboratory Press, Cold Spring Harbor, N.Y., (1988)].

Various procedures known in the art may be used for the production of polyclonal antibodies to a particular CPV-2c capsid protein, variants or derivatives or analogs thereof. For the production of an antibody, various host animals can be immunized by injection with the CPV-2c capsid protein, variant or a derivative (e.g., or fusion protein) thereof or fragment thereof, including but not limited to rabbits, mice, rats, sheep, goats, etc. In one embodiment, the inventive protein can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, and dinitrophenol.

For preparation of monoclonal antibodies directed toward a given inventive protein, variant, or analog, or derivative thereof, any technique that provides for the production of antibody molecules by continuous cell lines in culture may be used. These include but are not limited to the hybridoma technique originally developed by Kohler and Milstein [Nature, 256:495-497 (1975)], as well as the trioma technique, and the human B cell hybridoma technique [Kozbor et al., Immunology Today, 4:72 (1983); Cote et al., Proc. Natl. Acad Sci. U.S.A., 80:2026-2030 (1983)].

The monoclonal antibodies of the present invention include chimeric antibodies versions of antibodies originally produced in mice or other non-human animals. Techniques developed for the production of “chimeric antibodies” by splicing the genes from a mouse antibody molecule specific for a given inventive protein, for example, together with genes from a canine antibody of appropriate biological activity can be used. Such chimeric antibodies are within the scope of this invention [see in general, Morrison et al., J Bacteriol, 159:870 (1984); Neuberger et al., Nature, 312:604-608 (1984); Takeda et al., Nature, 314:452-454 (1985)].

### Kits

The present invention further comprises kits that are suitable for use in performing the methods described above. The kit may comprise a dosage form comprising a vaccine described above. The kit also may comprise at least one additional component, and, typically, instructions for using the vaccine with the additional component(s). The additional component(s) may, for example, be one or more additional ingredients (such as, for example, one or more of the excipients discussed above, food, and/or a treat) that can be mixed with the vaccine before or during administration. The additional component(s) may alternatively (or additionally) comprise one or more apparatuses for administering the vaccine to the canine or feline subject. Such an apparatus may be, for example, a syringe, a supralingual applicator, inhaler, nebulizer, pipette, forceps, or any medically acceptable delivery vehicle. In some embodiments, the apparatus is suitable for subcutaneous administration of the vaccine. In some embodiments, the apparatus is suitable for intranasal administration of the vaccine.

The present invention may be better understood by reference to the following nonlimiting Examples, which are provided as exemplary of the invention. The following examples are presented in order to more fully illustrate the preferred embodiments of the invention. They should in no way be construed, however, as limiting the broad scope of the invention.

### EXAMPLES

### Example 1

### Isolation and Attenuation of a Novel CPV Isolate

Two canine parvoviruses were selected (Isolates #4 and #12) from a group of isolates obtained from canine intestinal samples submitted to Oklahoma Animal Disease Diagnostic Laboratories (OADDL). The viruses were identified as being CPV-2c isolates by sequence analysis of their respective VP2 proteins. Initial studies showed that the selected CPV-2c isolates induced severe parvovirus disease in puppies. To attenuate their virulence, the viruses were serially passaged approximately forty times on Crandell feline kidney cells (CrFK), followed by a minimum of four additional passages on feline embryonic fibroblast (FEF) cells. The cells were grown on Eagle's minimal essential medium (EMEM) with 5 to 10% fetal bovine serum. During the in vitro attenuation process, the viruses were subjected to limited dilution cloning after every 10^{th} passage.

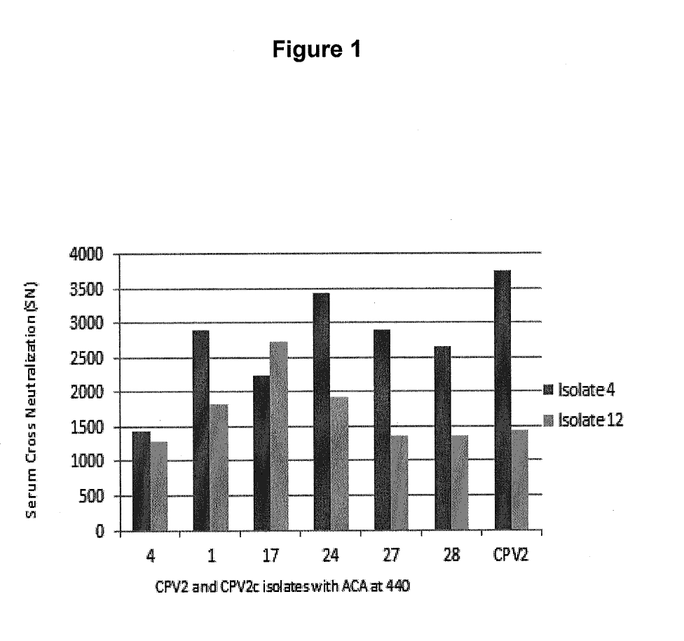

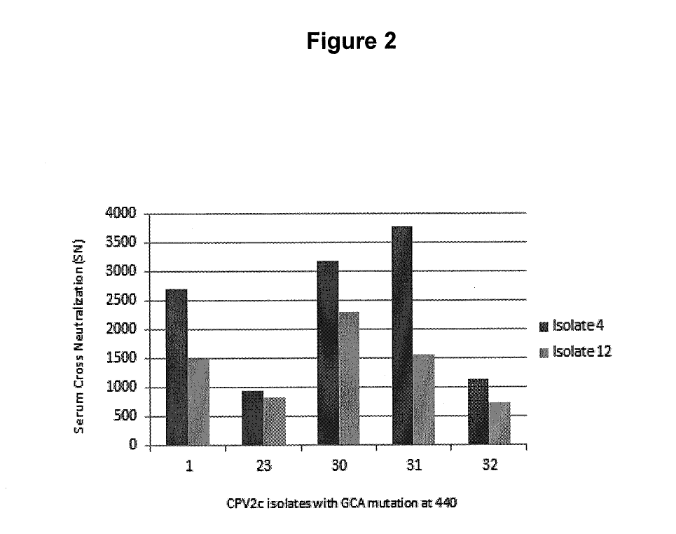

Initial cross-neutralization studies with serum from dogs inoculated with CPV-2c isolates #4 and #12 demonstrated that attenuated isolate #4 induced higher levels of cross neutralization antibodies compared to the attenuated isolate #12 (see, FIGS. 1 and 2). Indeed, attenuated CPV-2c isolate #4 (ATCC accession No. PTA-13492) was found to have superior serum neutralization properties when tested against either a standard CPV-2 isolate or against a variety of CPV-2c field isolates, including those having the A₄₄₀ modification [U.S. Pat. No. 8,227,583 B2; U.S. Pat. No. 8,258,274 B2].

The amino acid sequence of the VP2 capsid protein of the attenuated CPV-2c isolate #4 has six (6) amino acid residue modifications relative to that of the corresponding publicly available consensus amino acid sequences for CPV-2c VP2: a lysine residue at position 93 (K₉₃) in place of an asparagine residue, a lysine residue at position 219 (K₂₁₉) in place of an isoleucine residue, a lysine residue at position 377 (K₃₇₇) in place of an arginine residue, an isoleucine residue at position 555 (I₅₅₅) in place of a valine residue, a serine residue at position 300 (S₃₀₀) in place of a glycine residue, and an alanine residue at position 301 (A₃₀₁) in place of a threonine residue. All six of these modifications appear to be unique for this particular CPV-2c isolate, though at least five of the six the amino acid sites had been noted earlier for FPV and/or earlier CPV variants. The affect of these amino acid substitutions are provided in Table 1. In addition, the overall charge of the VP2 protein of the CPV-2c isolate #4 has been altered due to two of these amino acid changes involving the exchange of a neutral amino acid residue with a lysine residue, which is a basic amino acid.

The change of a lysine residue at position 93 (K₉₃) to an asparagine residue (N₉₃), along with the change of an aspartic acid residue at position 323 (D₃₂₃) to an asparagine residue (N₃₂₃) enabled FPV to bind the host cell canine transferrin receptor and infect canine cells. Therefore, it would appear that the presence of an arginine residue at position 93 in place of a lysine residue enhances the binding of a canine parvovirus isolate to its host cell receptor. Accordingly, substituting a lysine residue at position 93 (K₉₃) for an asparagine residue, as is found in the VP2 protein of isolate #4, would be expected to adversely affect the ability of that isolate to bind its host cell receptor. Similarly, changing an arginine residue at position 377 to a lysine residue (K₃₇₇) of the VP2 protein had eliminated the ability of an earlier CPV variant to bind erythrocytes (see, Table 1 below). Therefore, the analogous amino acid exchange in CPV-2c, as is found in the VP2 protein of isolate #4, also would be expected to adversely affect the ability of that isolate to bind erythrocytes.

Changing the glycine residue at position 300 (G₃₀₀) to a serine residue (S₃₀₀) of the VP2 protein might also be expected to have an adverse affect on isolate #4 to bind to its host cell receptor. In this regard, a CPV-2 isolate that had an aspartic acid at position 300 (D₃₀₀) was reported to be unable to either bind the host canine transferrin receptor or to infect canine cells or dogs. On the other hand, the change of an alanine residue (A₃₀₀) to a glycine residue (G₃₀₀), which occurred when CPV-2 mutated to CPV-2a, does not appear to have adversely affected the binding of the new variant to the host cell receptor. Interestingly, the glycine residue at position 300 of the wild type CPV-2c VP2 protein differs from the alanine at position 301 of isolate #4 by only the addition of a methyl group (CH₃) of the alanine, whereas the wild type threonine at position 301 of the VP2 protein differs only by the loss of a methylene group (—CH₂—) relative to the serine at position 300 of isolate #4. Therefore, the changes at these two adjacent amino acid residues may simply complement each other.

The lysine residue at position 219 (K₂₁₉) of the VP2 protein of the CPV-2c isolate #4 may aid in its attenuation, since a change of the isoleucine residue to a valine residue at position 219 in a recombinant heterogenous canine parvovirus enhanced the attenuation of that recombinant parvovirus when it was performed in conjunction with a concomitant change at position 386 of a glutamine residue to a lysine residue [WO2011107534 (A1); WO2012007589 (A1)]. Finally, the change of the valine residue to an isoleucine at position 555 (I₅₅₅) in the VP2 protein of the CPV-2c isolate #4 appears to be simply the third reiteration of a cyclical reversion.

TABLE 1
Amino Acid Changes in VP2 Capsid Protein of CPV-2c Isolate

| AA | From | To | Related Functional Changes |
|---|---|---|---|
| 93 | Asn | Lys | Changing a lysine residue (K₉₃) to an asparagine |
| | | | residue (N₉₃) enables FPV to bind the canine |
| | | | transferrin receptor when the aspartic acid residue |
| | | | at position 323 (D₃₂₃) is changed to an asparagine |
| | | | (N₃₂₃) as well. |
| 219 | Ile | Lys | Changing an isoleucine residue (I₂₁₉) to a |
| | | | valine residue of a CPV-2c VP2 protein (V₂₁₉) |
| | | | attenuates a CPV-2c-CPV-2 chimeric virus, |
| | | | when the glutamine residue at position 386 |
| | | | (Q₃₈₆) is changed to a lysine residue (K₃₈₆) |
| | | | as well. |
| 300 | Gly | Ser | One difference between the VP2 protein of |
| | | | CPV-2 and subsequent CPV variants, is that |
| | | | CPV-2 has an alanine residue (A₃₀₀) at |
| | | | positiin 300, whereas the later variants all have |
| | | | a glycine residue (G₃₀₀) in its place. However, |
| | | | when that same alanine residue (A₃₀₀) of |
| | | | CPV-2 was changed to an aspartic acid |
| | | | residue (D₃₀₀), CPV-2 was prevented from |
| | | | binding the host canine transferrin receptor |
| | | | and from infecting canine cells or dogs. |
| 301 | Thr | Ala | — |
| 377 | Arg | Lys | Changing an arginine residue (R₃₇₇) to |
| | | | a lysine residue (K₃₇₇) in CPV-2 |
| | | | eliminated the ability of the |
| | | | parvovirus to bind erythrocytes. |
| 555 | Val | Ile | CPV-2 → CPV-2a → CPV-2b → |
| | | | CPV-2c → CPV-2c (#4) |
| | | | Val → Ile → Val = Val → Ile |

The nucleotide sequences (SEQ ID NOs: 1, 3, 5, 7, and 9) and the amino acid sequences (SEQ ID NOs: 2, 4, 6, and 8) of the CPV-2c isolate (ATCC accession No. PTA-13492) are provided below:

| The Nucleotide Sequence that encodes the VP2 protein | |
|---|---|
| (SEQ ID NO: 1) | |
| 1 | ATGAGTGATG GAGCAGTTCA ACCAGACGGT GGTCAACCTG CTGTCAGAAA TGAAAGAGCT ACAGGATCTG GGAACGGGTC |
| | TGGAGGCGGG GGTGGTGGTG |
| 101 | GTTCTGGGGG TGTGGGGATT TCTACGGGTA CTTTTAATAA TCAGACGGAA TTTAAATTTT TGGAAAACGG ATGGGTGGAA |
| | ATCACAGCAA ACTCAAGCAG |
| 201 | ACTTGTACAT TTAAATATGC CAGAAAGTGA AAATTATAGA AGAGTGGTTG TAAATAATTT GGATAAAACT GCAGTTAAAG |
| | GAAACATGGC TTTAGATGAT |
| 301 | ACTCATGCAC AAATTGTAAC ACCTTGGTCA TTGGTTGATG CAAATGCTTG GGGAGTTTGG TTTAATCCAG GAGATTGGCA |
| | ACTAATTGTT AATACTATGA |
| 401 | GTGAGTTGCA TTTAGTTAGT TTTGAACAAG AAATTTTTAA TGTTGTTTTA AAGACTGTTT CAGAATCTGC TACTCAGCCA |
| | CCAACTAAAG TTTATAATAA |
| 501 | TGATTTAACT GCATCATTGA TGGTTGCATT AGATAGTAAT AATACTATGC CATTTACTCC AGCAGCTATG AGATCTGAGA |
| | CATTGGGTTT TTATCCATGG |
| 601 | AAACCAACCA TACCAACTCC ATGGAGATAT TATTTTCAAT GGGATAGAAC ATTAAAACCA TCTCATACTG GAACTAGTGG |
| | CACACCAACA AATATATACC |
| 701 | ATGGTACAGA TCCAGATGAT GTTCAATTTT ATACTATTGA AAATTCTGTG CCAGTACACT TACTAAGAAC AGGTGATGAA |
| | TTTGCTACAG GAACATTTTT |
| 801 | TTTTGATTGT AAACCATGTA GACTAACACA TACATGGCAA ACAAATAGAG CATTGGGCTT ACCACCATTT CTAAATTCTT |
| | TGCCTCAAGC TGAAGGAAGT |
| 901 | GCTAACTTTG GTTATATAGG AGTTCAACAA GATAAAAGAC GTGGTGTAAC TCAAATGGGA AATACAAACT ATATTACTGA |
| | AGCTACTATT ATGAGACCAG |
| 1001 | CTGAGGTTGG TTATAGTGCA CCATATTATT CTTTTGAGGC GTCTACACAA GGGCCATTTA AAACACCTAT TGCAGCAGGA |
| | CGGGGGGGAG CGCAAACAGA |
| 1101 | TGAAAATCAA GCAGCAGATG GTGATCCAAA ATATGCATTT GGTAGACAAC ATGGTCAAAA AACTACCACA ACAGGAGAAA |
| | CACCTGAGAG ATTTACATAT |
| 1201 | ATAGCACATC AAGATACAGG AAGATATCCA GAAGGAGATT GGATTCAAAA TATTAACTTT AACCTTCCTG TAACAGAAGA |
| | TAATGTATTG CTACCAACAG |
| 1301 | ATCCAATTGG AGGTAAAACA GGAATTAACT ATACTAATAT ATTTAATACT TATGGTCCTT TAACTGCATT AAATAATGTA |
| | CCACCAGTTT ATCCAAATGG |
| 1401 | TCAAATTTGG GATAAAGAAT TTGATACTGA CTTAAAACCA AGACTTCATG TAAATGCACC ATTTGTTTGT CAAAATAATT |
| | GTCCTGGTCA ATTATTTGTA |
| 1501 | AAAGTTGCGC CTAATTTAAC AAATGAATAT GATCCTGATG CATCTGCTAA TATGTCAAGA ATTGTAACTT ACTCAGATTT |
| | TTGGTGGAAA GGTAAATTAG |
| 1601 | TATTTAAAGC TAAACTAAGA GCCTCTCATA CTTGGAATCC AATTCAACAA ATGAGTATTA ATATAGATAA CCAATTTAAC |
| | TATGTACCAA GTAATATTGG |
| 1701 | AGGTATGAAA ATTGTATATG AAAAATCTCA ACTAGCACCT AGAAAATTAT ATTAA |
| The Amino Acid Sequence of the VP2 protein | |
| (SEQ ID NO: 2) | |
| 1 | MSDGAVQPDG GQPAVRNERA TGSGNGSGGG GGGGSGGVGI STGTFNNQTE |
| 51 | FKFLENGWVE ITANSSRLVH LNMPESENYR RVVVNNLDKT AVKGNMALDD |
| 101 | THAQIVTPWS LVDANAWGVW FNPGDWQLIV NTMSELHLVS FEQEIFNVVL |
| 151 | KTVSESATQP PTKVYNNDLT ASLMVALDSN NTMPFTPAAM RSETLGFYPW |
| 201 | KPTIPTPWRY YFQWDRTLKP SHTGTSGTPT NIYHGTDPDD VQFYTIENSV |
| 251 | PVHLLRTGDE FATGTFFFDC KPCRLTHTWQ TNRALGLPPF LNSLPQAEGS |
| 301 | ANFGYIGVQQ DKRRGVTQMG NTNYITEATI MRPAEVGYSA PYYSFEASTQ |
| 351 | GPFKTPIAAG RGGAQTDENQ AADGDPKYAF GRQHGQKTTT TGETPERFTY |
| 401 | IAHQDTGRYP EGDWIQNINF NLPVTEDNVL LPTDPIGGKT GINYTNIFNT |
| 451 | YGPLTALNNV PPVYPNGQIW DKEFDTDLKP RLHVNAPFVC QNNCPGQLFV |
| 501 | KVAPNLTNEY DPDASANMSR IVTYSDFWWK GKLVFKAKLR ASHTWNPIQQ |
| 551 | MSINIDNQFN YVPSNIGGMK IVYEKSQLAP RKLY* |
| The Nucleotide Sequence that encodes the VP1 protein | |
| (SEQ ID NO: 3) | |
| 1 | ATGGCACCTC CGGCAAAGAG AGCCAGGAGA GGACTTGTGC CTCCAGGTTA TAAATATCTT GGGCCTGGGA ACAGTCTTGA |
| | CCAAGGAGAA CCAACTAACC |
| 101 | CTTCTGACGC CGCTGCAAAA GAACACGACG AAGCTTACGC TGCTTATCTT CGCTCTGGTA AAAACCCATA CTTATATTTC |
| | TCGCCAGCAG ATCAACGCTT |
| 201 | TATAGATCAA ACTAAGGACG CTAAAGATTG GGGGGGGAAA ATAGGACATT ATTTTTTTAG AGCTAAAAAG GCAATTGCTC |
| | CAGTATTAAC TGATACACCA |
| 301 | GATCATCCAT CAACATCAAG ACCAACAAAA CCAACTAAAA GAAGTAAACC ACCACCTCAT ATTTTCATCA ATCTTGCAAA |
| | AAAAAAAAAA GCCGGTGCAG |
| 401 | GACAAGTAAA AAGAGACAAT CTTGCACCAA TGAGTGATGG AGCAGTTCAA CCAGACGGTG GTCAACCTGC TGTCAGAAAT |
| | GAAAGAGCTA CAGGATCTGG |
| 501 | GAACGGGTCT GGAGGCGGGG GTGGTGGTGG TTCTGGGGGT GTGGGGATTT CTACGGGTAC TTTTAATAAT CAGACGGAAT |
| | TTAAATTTTT GGAAAACGGA |
| 601 | TGGGTGGAAA TCACAGCAAA CTCAAGCAGA CTTGTACATT TAAATATGCC AGAAAGTGAA AATTATAGAA GAGTGGTTGT |
| | AAATAATTTG GATAAAACTG |
| 701 | CAGTTAAAGG AAACATGGCT TTAGATGATA CTCATGCACA AATTGTAACA CCTTGGTCAT TGGTTGATGC AAATGCTTGG |
| | GGAGTTTGGT TTAATCCAGG |
| 801 | AGATTGGCAA CTAATTGTTA ATACTATGAG TGAGTTGCAT TTAGTTAGTT TTGAACAAGA AATTTTTAAT GTTGTTTTAA |
| | AGACTGTTTC AGAATCTGCT |
| 901 | ACTCAGCCAC CAACTAAAGT TTATAATAAT GATTTAACTG CATCATTGAT GGTTGCATTA GATAGTAATA ATACTATGCC |
| | ATTTACTCCA GCAGCTATGA |
| 1001 | GATCTGAGAC ATTGGGTTTT TATCCATGGA AACCAACCAT ACCAACTCCA TGGAGATATT ATTTTCAATG GGATAGAACA |
| | TTAAAACCAT CTCATACTGG |
| 1101 | AACTAGTGGC ACACCAACAA ATATATACCA TGGTACAGAT CCAGATGATG TTCAATTTTA TACTATTGAA AATTCTGTGC |
| | CAGTACACTT ACTAAGAACA |
| 1201 | GGTGATGAAT TTGCTACAGG AACATTTTTT TTTGATTGTA AACCATGTAG ACTAACACAT ACATGGCAAA CAAATAGAGC |
| | ATTGGGCTTA CCACCATTTC |
| 1301 | TAAATTCTTT GCCTCAAGCT GAAGGAAGTG CTAACTTTGG TTATATAGGA GTTCAACAAG ATAAAAGACG TGGTGTAACT |
| | CAAATGGGAA ATACAAACTA |
| 1401 | TATTACTGAA GCTACTATTA TGAGACCAGC TGAGGTTGGT TATAGTGCAC CATATTATTC TTTTGAGGCG TCTACACAAG |
| | GGCCATTTAA AACACCTATT |
| 1501 | GCAGCAGGAC GGGGGGGAGC GCAAACAGAT GAAAATCAAG CAGCAGATGG TGATCCAAAA TATGCATTTG GTAGACAACA |
| | TGGTCAAAAA ACTACCACAA |
| 1601 | CAGGAGAAAC ACCTGAGAGA TTTACATATA TAGCACATCA AGATACAGGA AGATATCCAG AAGGAGATTG GATTCAAAAT |
| | ATTAACTTTA ACCTTCCTGT |
| 1701 | AACAGAAGAT AATGTATTGC TACCAACAGA TCCAATTGGA GGTAAAACAG GAATTAACTA TACTAATATA TTTAATACTT |
| | ATGGTCCTTT AACTGCATTA |
| 1801 | AATAATGTAC CACCAGTTTA TCCAAATGGT CAAATTTGGG ATAAAGAATT TGATACTGAC TTAAAACCAA GACTTCATGT |
| | AAATGCACCA TTTGTTTGTC |
| 1901 | AAAATAATTG TCCTGGTCAA TTATTTGTAA AAGTTGCGCC TAATTTAACA AATGAATATG ATCCTGATGC ATCTGCTAAT |
| | ATGTCAAGAA TTGTAACTTA |
| 2001 | CTCAGATTTT TGGTGGAAAG GTAAATTAGT ATTTAAAGCT AAACTAAGAG CCTCTCATAC TTGGAATCCA ATTCAACAAA |
| | TGAGTATTAA TATAGATAAC |
| 2101 | CAATTTAACT ATGTACCAAG TAATATTGGA GGTATGAAAA TTGTATATGA AAAATCTCAA CTAGCACCTA |
| | GAAAATTATA TTAA |
| The Amino Acid Sequence of the VP1 protein | |
| (SEQ ID NO: 4) | |
| 1 | MAPPAKRARR GLVPPGYKYL GPGNSLDQGE PTNPSDAAAK EHDEAYAAYL |
| 51 | RSGKNPYLYF SPADQRFIDQ TKDAKDWGGK IGHYFFRAKK AIAPVLTDTP |
| 101 | DHPSTSRPTK PTKRSKPPPH IFINLAKKKK AGAGQVKRDN LAPMSDGAVQ |
| 151 | PDGGQPAVRN ERATGSGNGS GGGGGGGSGG VGISTGTFNN QTEFKFLENG |
| 201 | WVEITANSSR LVHLNMPESE NYRRVVVNNL DKTAVKGNMA LDDTHAQIVT |
| 251 | PWSLVDANAW GVWFNPGDWQ LIVNTMSELH LVSFEQEIFN VVLKTVSESA |
| 301 | TQPPTKVYNN DLTASLMVAL DSNNTMPFTP AAMRSETLGF YPWKPTIPTP |
| 351 | WRYYFQWDRT LKPSHTGTSG TPTNIYHGTD PDDVQFYTIE NSVPVHLLRT |
| 401 | GDEFATGTFF FDCKPCRLTH TWQTNRALGL PPFLNSLPQA EGSANFGYIG |
| 451 | VQQDKRRGVT QMGNTNYITE ATIMRPAEVG YSAPYYSFEA STQGPFKTPI |
| 501 | AAGRGGAQTD ENQAADGDPK YAFGRQHGQK TTTTGETPER FTYIAHQDTG |
| 551 | RYPEGDWIQN INFNLPVTED NVLLPTDPIG GKTGINYTNI FNTYGPLTAL |
| 601 | NNVPPVYPNG QIWDKEFDTD LKPRLHVNAP FVCQNNCPGQ LFVKVAPNLT |
| 651 | NEYDPDASAN MSRIVTYSDF WWKGKLVFKA KLRASHTWNP IQQMSINIDN |
| 701 | QFNYVPSNIG GMKIVYEKSQ LAPRKLY* |
| The Nucleotide Sequence that encodes the NS2 protein | |
| (SEQ ID NO: 5) | |
| 1 | ATGTCTGGCA ACCAGTATAC TGAGGAAGTT ATGGAGGGAG TAAATTGGTT AAAGAAACAT GCAGAGAATG AAGCATTTTC |
| | GTTTGTTTTT AAATGTGACA |
| 101 | ACGTCCAACT AAATGGAAAG GATGTTCGCT GGAACAACTA TACCAAACCA ATTCAAAATG AAGAGCTAAC ATCTTTAATT |
| | AGAGGAGCAC AAACAGCAAT |
| 201 | GGATCAAACC GAAGAAGAAG AAATGGACTG GGAATCGGAA GTTGATAGTC TCGCCAAAAA GTTGCAAAGA CTTAGAGACG |
| | CAAGCGGCAA GCAATCCTCA |
| 301 | GAGTCAAGAC CAAGCTCTAA CTCCTCTGAC TCCGAACGTA GTGGACCTTG CACTGGAACC GTGGAGTACT CCAGATACGC |
| | CTATTGCAAA AACTGCAAAT |
| 401 | CAACAATCAA ACCAACTTGG CGTTACTCAC AAAGACGTGC AAGCGAGTCC AACGTGGTCC GAAATAGAGG CAGACCTGAG |
| | AGCCATCTTT ACTTCTGA |
| The Amino Acid Sequence of the NS2 protein | |
| (SEQ ID NO: 6) | |
| 1 | MSGNQYTEEV MEGVNWLKKH AENEAFSFVF KCDNVQLNGK DVRWNNYTKP |
| 51 | IQNEELTSLI RGAQTAMDQT EEEEMDWESE VDSLAKKLQR LRDASGKQSS |
| 101 | ESRPSSNSSD SERSGPCTGT VEYSRYAYCK NCKSTIKPTW RYSQRRASES |
| 151 | NVVRNRGRPE SHLYF* |
| The Nucleotide Sequence that encodes the NS1 protein | |
| (SEQ ID NO: 7) | |
| 1 | ATGTCTGGCA ACCAGTATAC TGAGGAAGTT ATGGAGGGAG TAAATTGGTT AAAGAAACAT GCAGAGAATG AAGCATTTTC |
| | GTTTGTTTTT AAATGTGACA |
| 101 | ACGTCCAACT AAATGGAAAG GATGTTCGCT GGAACAACTA TACCAAACCA ATTCAAAATG AAGAGCTAAC ATCTTTAATT |
| | AGAGGAGCAC AAACAGCAAT |
| 201 | GGATCAAACC GAAGAAGAAG AAATGGACTG GGAATCGGAA GTTGATAGTC TCGCCAAAAA GCAAGTACAA ACTTTTGATG |
| | CATTAATTAA AAAATGTCTT |
| 301 | TTTGAAGTCT TTGTTTCTAA AAATATAGAA CCAAATGAAT GTGTTTGGTT TATTCAACAT GAATGGGGAA AAGATCAAGG |
| | CTGGCATTGT CATGTTTTAC |
| 401 | TTCATAGTAA GAACTTACAA CAAGCAACTG GTAAATGGCT ACGCAGACAA ATGAATATGT ATTGGAGTAG ATGGTTGGTG |
| | ACTCTTTGTT CGGTAAATTT |
| 501 | AACACCAACT GAAAAGATTA AGCTCAGAGA AATTGCAGAA GATAGTGAAT GGGTGACTAT ATTAACATAC AGACATAAGC |
| | AAACAAAAAA AGACTATGTT |
| 601 | AAAATGGTTC ATTTTGGAAA TATGATAGCA TATTACTTTT TAACAAAGAA AAAAATTGTC CACATGACAA AAGAAAGTGG |
| | CTATTTTTTA AGTACTGATT |
| 701 | CTGGTTGGAA ATTTAACTTT ATGAAGTATC AAGACAGACA AATTGTCAGC ACACTTTACA CTGAACAAAT GAAACCAGAA |
| | ACCGTTGAAA CCACAGTGAC |
| 801 | GACAGCACAG GAAACAAAGC GCGGGAGAAT TCAAACTAAA AAGGAAGTAT CAATCAAATG TACTTTGCGG GACTTGGTTA |
| | GTAAAAGAGT AACATCACCT |
| 901 | GAAGACTGGA TGATGTTACA ACCAGATAGT TATATTGAAA TGATGGCACA ACCAGGAGGT GAAAATCTTT TAAAAAATAC |
| | ACTTGAAATT TGTACTTTGA |
| 1001 | CTTTAGCAAG AACAAAAACA GCATTTGAAT TAATACTTGA AAAAGCAGAT AATACTAAAC TGACTAACTT TGATCTTGCA |
| | AATTCTAGAA CATGTCAGAT |
| 1101 | TTTTAGAATG CACGGATGGA ATTGGATTAA AGTTTGTCAC GCTATAGCAT GTGTTTTAAA TAGACAAGGT GGTAAAAGAA |
| | ATACAGTTCT TTTTCATGGA |
| 1201 | CCAGCAAGTA CAGGAAAATC TATCATTGCT CAAGCCATAG CACAAGCTGT GGGTAATGTT GGTTGTTATA ATGCAGCAAA |
| | TGTAAATTTT CCATTTAATG |
| 1301 | ACTGCACCAA TAAAAATTTA ATTTGGATTG AAGAAGCTGG TAACTTTGGT CAACAAGTTA ATCAATTTAA AGCAATTTGT |
| | TCCGGACAAA CAATTAGAAT |
| 1401 | TGATCAAAAA GGTAAAGGAA GTAAGCAAAT TGAACCAACT CCAGTAATTA TGACAACTAA TGAAAATATA ACAATTGTGA |
| | GAATTGGATG TGAAGAAAGA |
| 1501 | CCTGAACATA CACAACCAAT AAGAGACAGA ATGTTGAACA TTAAGTTAGT ATGTAAGCTT CCAGGAGACT TTGGTTTGGT |
| | TGATAAAGAA GAATGGCCTT |
| 1601 | TAATATGTGC ATGGTTAGTT AAACATGGTT ATGAATCAAC CATGGCTAAC TATACACATC ATTGGGGAAA AGTACCAGAA |
| | TGGGATGAAA ACTGGGCGGA |
| 1701 | GCCTAAAATA CAAGAAGGTA TAAATTCACC AGGTTGCAAA GACTTAGAGA CGCAAGCGGC AAGCAATCCT CAGAGTCAAG |
| | ACCAAGCTCT AACTCCTCTG |
| 1801 | ACTCCGAACG TAGTGGACCT TGCACTGGAA CCGTGGAGTA CTCCAGATAC GCCTATTGCA AAAACTGCAA ATCAACAATC |
| | AAACCAACTT GGCGTTACTC |
| 1901 | ACAAAGACGT GCAAGCGAGT CCAACGTGGT CCGAAATAGA GGCAGACCTG AGAGCCATCT TTACTTCTGA ACAACTGGAA |
| | GAAGATTTTC AAGACGACTT |
| 2001 | GGATTAA |
| The Amino Acid Sequence of the NS1 protein | |
| (SEQ ID NO: 8) | |
| 1 | MSGNQYTEEV MEGVNWLKKH AENEAFSFVF KCDNVQLNGK DVRWNNYTKP |
| 51 | IQNEELTSLI RGAQTAMDQT EEEEMDWESE VDSLAKKQVQ TFDALIKKCL |
| 101 | FEVFVSKNIE PNECVWFIQH EWGKDQGWHC HVLLHSKNLQ QATGKWLRRQ |
| 151 | MNMYWSRWLV TLCSVNLTPT EKIKLREIAE DSEWVTILTY RHKQTKKDYV |
| 201 | KMVHFGNMIA YYFLTKKKIV HMTKESGYFL STDSGWKFNF MKYQDRQIVS |
| 251 | TLYTEQMKPE TVETTVTTAQ ETKRGRIQTK KEVSIKCTLR DLVSKRVTSP |
| 301 | EDWMMLQPDS YIEMMAQPGG ENLLKNTLEI CTLTLARTKT AFELILEKAD |
| 351 | NTKLTNFDLA NSRTCQIFRM HGWNWIKVCH AIACVLNRQG GKRNTVLFHG |
| 401 | PASTGKSIIA QAIAQAVGNV GCYNAANVNF PFNDCTNKNL IWIEEAGNFG |
| 451 | QQVNQFKAIC SGQTIRIDQK GKGSKQIEPT PVIMTTNENI TIVRIGCEER |
| 501 | PEHTQPIRDR MLNIKLVCKL PGDFGLVDKE EWPLICAWLV KHGYESTMAN |
| 551 | YTHHWGKVPE WDENWAEPKI QEGINSPGCK DLETQAASNP QSQDQALTPL |
| 601 | TPNVVDLALE PWSTPDTPIA KTANQQSNQL GVTHKDVQAS PTWSEIEADL |
| 651 | RAIFTSEQLE EDFQDDLD* |
| The Nucleotide Sequence for the entire genome missing a small portion of the 3′ end | |
| (SEQ ID NO: 9) | |
| 1 | ATCATTCTTT AGAACCAACT GACCAAGTTC ACGTACGTAT GACGTGATGA CGCGCGCTGC GCGCGCTGCC |
| | TACGGCAGTC ACACGTCATA CGTACGCTCC |
| 101 | TTGGTCAGTT GGTTCTAAAG AATGATAGGC GGTTTGTGTG TTTAAACTTG GGCGGGAAAA GGTGGCGGGC |
| | TAATTGTGGG CGTGGTTAAA GGTATAAAAG |
| 201 | ACAAACCATA GACCGTTACT GACATTCGCT TCTTGTCTTT GACAGAGTGA ACCTCTCTTA CTTTGACTAA |
| | CCATGTCTGG CAACCAGTAT ACTGAGGAAG |
| 301 | TTATGGAGGG AGTAAATTGG TTAAAGAAAC ATGCAGAGAA TGAAGCATTT TCGTTTGTTT TTAAATGTGA |
| | CAACGTCCAA CTAAATGGAA AGGATGTTCG |
| 401 | CTGGAACAAC TATACCAAAC CAATTCAAAA TGAAGAGCTA ACATCTTTAA TTAGAGGAGC ACAAACAGCA |
| | ATGGATCAAA CCGAAGAAGA AGAAATGGAC |
| 501 | TGGGAATCGG AAGTTGATAG TCTCGCCAAA AAGCAAGTAC AAACTTTTGA TGCATTAATT AAAAAATGTC |
| | TTTTTGAAGT CTTTGTTTCT AAAAATATAG |
| 601 | AACCAAATGA ATGTGTTTGG TTTATTCAAC ATGAATGGGG AAAAGATCAA GGCTGGCATT GTCATGTTTT |
| | ACTTCATAGT AAGAACTTAC AACAAGCAAC |
| 701 | TGGTAAATGG CTACGCAGAC AAATGAATAT GTATTGGAGT AGATGGTTGG TGACTCTTTG TTCGGTAAAT |
| | TTAACACCAA CTGAAAAGAT TAAGCTCAGA |
| 801 | GAAATTGCAG AAGATAGTGA ATGGGTGACT ATATTAACAT ACAGACATAA GCAAACAAAA AAAGACTATG |
| | TTAAAATGGT TCATTTTGGA AATATGATAG |
| 901 | CATATTACTT TTTAACAAAG AAAAAAATTG TCCACATGAC AAAAGAAAGT GGCTATTTTT TAAGTACTGA |
| | TTCTGGTTGG AAATTTAACT TTATGAAGTA |
| 1001 | TCAAGACAGA CAAATTGTCA GCACACTTTA CACTGAACAA ATGAAACCAG AAACCGTTGA AACCACAGTG |
| | ACGACAGCAC AGGAAACAAA GCGCGGGAGA |
| 1101 | ATTCAAACTA AAAAGGAAGT ATCAATCAAA TGTACTTTGC GGGACTTGGT TAGTAAAAGA GTAACATCAC |
| | CTGAAGACTG GATGATGTTA CAACCAGATA |
| 1201 | GTTATATTGA AATGATGGCA CAACCAGGAG GTGAAAATCT TTTAAAAAAT ACACTTGAAA TTTGTACTTT |
| | GACTTTAGCA AGAACAAAAA CAGCATTTGA |
| 1301 | ATTAATACTT GAAAAAGCAG ATAATACTAA ACTGACTAAC TTTGATCTTG CAAATTCTAG AACATGTCAG |
| | ATTTTTAGAA TGCACGGATG GAATTGGATT |
| 1401 | AAAGTTTGTC ACGCTATAGC ATGTGTTTTA AATAGACAAG GTGGTAAAAG AAATACAGTT CTTTTTCATG |
| | GACCAGCAAG TACAGGAAAA TCTATCATTG |
| 1501 | CTCAAGCCAT AGCACAAGCT GTGGGTAATG TTGGTTGTTA TAATGCAGCA AATGTAAATT TTCCATTTAA |
| | TGACTGCACC AATAAAAATT TAATTTGGAT |
| 1601 | TGAAGAAGCT GGTAACTTTG GTCAACAAGT TAATCAATTT AAAGCAATTT GTTCCGGACA AACAATTAGA |
| | ATTGATCAAA AAGGTAAAGG AAGTAAGCAA |
| 1701 | ATTGAACCAA CTCCAGTAAT TATGACAACT AATGAAAATA TAACAATTGT GAGAATTGGA TGTGAAGAAA |
| | GACCTGAACA TACACAACCA ATAAGAGACA |
| 1801 | GAATGTTGAA CATTAAGTTA GTATGTAAGC TTCCAGGAGA CTTTGGTTTG GTTGATAAAG AAGAATGGCC |
| | TTTAATATGT GCATGGTTAG TTAAACATGG |
| 1901 | TTATGAATCA ACCATGGCTA ACTATACACA TCATTGGGGA AAAGTACCAG AATGGGATGA AAACTGGGCG |
| | GAGCCTAAAA TACAAGAAGG TATAAATTCA |
| 2001 | CCAGGTTGCA AAGACTTAGA GACGCAAGCG GCAAGCAATC CTCAGAGTCA AGACCAAGCT CTAACTCCTC |
| | TGACTCCGAA CGTAGTGGAC CTTGCACTGG |
| 2101 | AACCGTGGAG TACTCCAGAT ACGCCTATTG CAAAAACTGC AAATCAACAA TCAAACCAAC TTGGCGTTAC |
| | TCACAAAGAC GTGCAAGCGA GTCCAACGTG |
| 2201 | GTCCGAAATA GAGGCAGACC TGAGAGCCAT CTTTACTTCT GAACAACTGG AAGAAGATTT TCAAGACGAC |
| | TTGGATTAAG GTACGATGGC ACCTCCGGCA |
| 2301 | AAGAGAGCCA GGAGAGGTAA GGGTGTGTTA GTAAAGTGGG GGGAGGGGAA AGATTTAGTA ACTTAACTAA |
| | GTATGTGTTT TTTTATAGGA CTTGTGCCTC |
| 2401 | CAGGTTATAA ATATCTTGGG CCTGGGAACA GTCTTGACCA AGGAGAACCA ACTAACCCTT CTGACGCCGC |
| | TGCAAAAGAA CACGACGAAG CTTACGCTGC |
| 2501 | TTATCTTCGC TCTGGTAAAA ACCCATACTT ATATTTCTCG CCAGCAGATC AACGCTTTAT AGATCAAACT |
| | AAGGACGCTA AAGATTGGGG GGGGAAAATA |
| 2601 | GGACATTATT TTTTTAGAGC TAAAAAGGCA ATTGCTCCAG TATTAACTGA TACACCAGAT CATCCATCAA |
| | CATCAAGACC AACAAAACCA ACTAAAAGAA |
| 2701 | GTAAACCACC ACCTCATATT TTCATCAATC TTGCAAAAAA AAAAAAAGCC GGTGCAGGAC AAGTAAAAAG |
| | AGACAATCTT GCACCAATGA GTGATGGAGC |
| 2801 | AGTTCAACCA GACGGTGGTC AACCTGCTGT CAGAAATGAA AGAGCTACAG GATCTGGGAA CGGGTCTGGA |
| | GGCGGGGGTG GTGGTGGTTC TGGGGGTGTG |
| 2901 | GGGATTTCTA CGGGTACTTT TAATAATCAG ACGGAATTTA AATTTTTGGA AAACGGATGG GTGGAAATCA |
| | CAGCAAACTC AAGCAGACTT GTACATTTAA |
| 3001 | ATATGCCAGA AAGTGAAAAT TATAGAAGAG TGGTTGTAAA TAATTTGGAT AAAACTGCAG TTAAAGGAAA |
| | CATGGCTTTA GATGATACTC ATGCACAAAT |
| 3101 | TGTAACACCT TGGTCATTGG TTGATGCAAA TGCTTGGGGA GTTTGGTTTA ATCCAGGAGA TTGGCAACTA |
| | ATTGTTAATA CTATGAGTGA GTTGCATTTA |
| 3201 | GTTAGTTTTG AACAAGAAAT TTTTAATGTT GTTTTAAAGA CTGTTTCAGA ATCTGCTACT CAGCCACCAA |
| | CTAAAGTTTA TAATAATGAT TTAACTGCAT |
| 3301 | CATTGATGGT TGCATTAGAT AGTAATAATA CTATGCCATT TACTCCAGCA GCTATGAGAT CTGAGACATT |
| | GGGTTTTTAT CCATGGAAAC CAACCATACC |
| 3401 | AACTCCATGG AGATATTATT TTCAATGGGA TAGAACATTA AAACCATCTC ATACTGGAAC TAGTGGCACA |
| | CCAACAAATA TATACCATGG TACAGATCCA |
| 3501 | GATGATGTTC AATTTTATAC TATTGAAAAT TCTGTGCCAG TACACTTACT AAGAACAGGT GATGAATTTG |
| | CTACAGGAAC ATTTTTTTTT GATTGTAAAC |
| 3601 | CATGTAGACT AACACATACA TGGCAAACAA ATAGAGCATT GGGCTTACCA CCATTTCTAA ATTCTTTGCC |
| | TCAAGCTGAA GGAAGTGCTA ACTTTGGTTA |
| 3701 | TATAGGAGTT CAACAAGATA AAAGACGTGG TGTAACTCAA ATGGGAAATA CAAACTATAT TACTGAAGCT |
| | ACTATTATGA GACCAGCTGA GGTTGGTTAT |
| 3801 | AGTGCACCAT ATTATTCTTT TGAGGCGTCT ACACAAGGGC CATTTAAAAC ACCTATTGCA GCAGGACGGG |
| | GGGGAGCGCA AACAGATGAA AATCAAGCAG |
| 3901 | CAGATGGTGA TCCAAAATAT GCATTTGGTA GACAACATGG TCAAAAAACT ACCACAACAG GAGAAACACC |
| | TGAGAGATTT ACATATATAG CACATCAAGA |
| 4001 | TACAGGAAGA TATCCAGAAG GAGATTGGAT TCAAAATATT AACTTTAACC TTCCTGTAAC AGAAGATAAT |
| | GTATTGCTAC CAACAGATCC AATTGGAGGT |
| 4101 | AAAACAGGAA TTAACTATAC TAATATATTT AATACTTATG GTCCTTTAAC TGCATTAAAT AATGTACCAC |
| | CAGTTTATCC AAATGGTCAA ATTTGGGATA |
| 4201 | AAGAATTTGA TACTGACTTA AAACCAAGAC TTCATGTAAA TGCACCATTT GTTTGTCAAA ATAATTGTCC |
| | TGGTCAATTA TTTGTAAAAG TTGCGCCTAA |
| 4301 | TTTAACAAAT GAATATGATC CTGATGCATC TGCTAATATG TCAAGAATTG TAACTTACTC AGATTTTTGG |
| | TGGAAAGGTA AATTAGTATT TAAAGCTAAA |
| 4401 | CTAAGAGCCT CTCATACTTG GAATCCAATT CAACAAATGA GTATTAATAT AGATAACCAA TTTAACTATG |
| | TACCAAGTAA TATTGGAGGT ATGAAAATTG |
| 4501 | TATATGAAAA ATCTCAACTA GCACCTAGAA AATTATATTA ACATACTTAC TATGTTTTTA TGTTTATTAC |
| | ATATTATTTT AAGATTAATT AAATTACAGC |
| 4601 | ATAGAAATAT TGTACTTGTA CTTGATATAG GATTTAGAAG GTTTGTTATA TGGTATACAA TAACTGTAAG |
| | AAATAGAAGA ACATTTAGAT CATAGTTAGT |
| 4701 | AGTTTGTTTT GTAAAATGTA TTGTAAACCA TTAATGTATG TTGTTATGGT GTGGGTGGTT GGTTGGTTTG |
| | CCCTTAGAAT ATGTTAAGGA CCAAAAAAAA |
| 4801 | TCAATAAAAG ACATTTAAAA CTAAATGGCC TCGTATACTG TCTATAAGGT GAACTAACCT TACCATAAGT |
| | ATCAATCTGT CTTTAAGGGG GGGGTGGGTG |
| 4901 | GGAGATGNNN NNNNNNNNNN NNNNNNNNNN NNNNNNNNNN NNNNNNNNNN NNNNNNNNNN NNNNNNNNNN NNNNNNNNNN |
| | NNNNNNNNNN NNNNNNNNNN |
| 5001 | NNNNNNNNNN NNNNNNNNNN NNNNNNNNNN NNNNNNNNNN NNNNNNNNNN NNNNNNNN |

The complete Non-structural gene (NS0): 273-2279
The complete Capsid gene (VP0): 2286-4541
NS1 CDS: 273-2279
NS2 CDS: join 273-533 . . . 2006-2242
VP1 CDS: join 2286-2315 . . . 2388-4541
VP2 CDS: 2787-4541

### Example 2

### Efficacy of the Novel CPV-2c Vaccine Against a Virulent CPV-2b Challenge

The efficacy of a multivalent vaccine comprising the attenuated CPV-2c isolate #4 of the present invention in combination with a live canine distemper virus (CDV) isolate, a live canine adenovirus type 2 (CAV2) isolate, and a live canine parainfluenza virus (CPI) isolate was tested against a virulent CPV-2b challenge.

### Materials and Methods

Study Protocols: Prior to initiation of animal studies, all animal study protocols were reviewed and approved by Institutional Animal Care and Use Committee (IACUC).

Animals: Twenty-five 6 to 8 week old, specific pathogen free (SPF) puppies from a licensed breeder were randomly assigned to either test vaccine group (N=20, Group 1) or placebo group (N=5, Group 2). All puppies were tested and found free of CPV antibodies prior to vaccination.

Vaccines: A multivalent vaccine was formulated with CDV, CAV2, CPI antigens and CPV-2c isolate #4 (ATCC accession No. PTA-13492). All antigens in the vaccine are modified live viruses. The multivalent vaccine was then lyophilized using standard procedures. Each dose of the test vaccine contained approximately 4 log₁₀ TCID₅₀ of CPV-2c virus. A modified live vaccine also was formulated with the CDV, CAV2 and CPi antigens, but without CPV-2c. This placebo vaccine was also lyophilized.

Vaccination and challenge: The puppies were vaccinated with two doses (3 weeks apart) of either the test vaccine (Group 1) or the placebo vaccine (Group 2) by subcutaneous injection, and then were challenged with a virulent CPV-2b isolate following a standard protocol at 3 weeks post-second vaccination.

Clinical observations: All animal were observed for fever (≧103.4° C.), clinical signs which include diarrhea, vomiting, mucous or blood in feces, lymphopenia (≧50% reduction prior to pre-challenge level) and fecal shedding. A puppy was considered as being positive for parvovirus disease if it was positive for 3 of the 4 pathogenomonic signs: lymphopenia, fever, fecal shedding, and clinical signs.

### Results and Conclusion

Prior to the challenge with CPV-2b, all puppies receiving the test vaccine were positive for CPV SN antibodies following the 2^{nd} vaccination dose (>2800), whereas all puppies receiving the placebo vaccine were free of CPV SN antibodies (<2). Following the CPV-2b challenge, 100% of the puppies that had been administered the placebo vaccine were positive for parvovirus disease, whereas 100% of the puppies vaccinated with the vaccine comprising the CPV-2c isolate #4 (i.e., the test vaccine) were free of parvovirus disease.

TABLE 2
EFFICACY OF TEST VACCINE WITH CPV-2c ISOLATE #
4 FOLLOWING A CHALLENGE WITH VIRULENT CPV-2b.

| Treatment | | | | % Dogs |
|---|---|---|---|---|
| Group | No. of dogs | Vaccine | Challenge virus | protected |
| 1 | 20 | Test Vaccine | CPV-2b | 100 |
| 2 | 5 | Placebo | CPV-2b | 0 |

The dogs were vaccinated subcutaneously with either two doses of the Test vaccine or two doses of the placebo prior to the challenge with CPV-2b

The results from this study (Table 2 above) demonstrate that the vaccine comprising the CPV-2c isolate #4 (ATCC accession No. PTA-13492) provided 100% protection against a virulent CPV-2b isolate. Indeed, although completely avirulent in dogs, the attenuated canine parvovirus of the present invention can still induce significant levels of protection against a CPV-2b challenge.

### Example 3

### Efficacy of the Novel CPV-2c Vaccine Against a Virulent CPV-2c Challenge

The efficacy of a multivalent vaccine comprising the attenuated CPV-2c isolate #4 of the present invention in combination with a live canine distemper virus (CDV) isolate, a live canine adenovirus type 2 (CAV2) isolate, and a live canine parainfluenza virus (CPI) isolate was tested against a virulent CPV-2c challenge.

### Materials and Methods

Study Protocols: Prior to initiation of animal studies, all animal study protocols were reviewed and approved by Institutional Animal Care and Use Committee (IACUC).

Animals: Twenty-five 6 to 8 week old, specific pathogen free (SPF) puppies from a licensed breeder were randomly assigned to either test vaccine group (N=20, Group 1) or placebo group (N=5, Group 2). All puppies were tested and found free of CPV antibodies prior to vaccination.

Vaccines: A multivalent vaccine was formulated with CDV, CAV2, CPI antigens and CPV-2c isolate #4 (ATCC accession No. PTA-13492). All antigens in the vaccine are modified live viruses. The multivalent vaccine was then lyophilized using standard procedures. Each dose of the test vaccine contained approximately 4 log₁₀ TCID₅₀ of CPV-2c virus. A modified live vaccine also was formulated with the CDV, CAV2 and CPi antigens, but without CPV-2c. This placebo vaccine was also lyophilized.

Vaccination and challenge: The puppies were vaccinated with two doses (3 weeks apart) of either the test vaccine (Group 1) or the placebo vaccine (Group 2) by subcutaneous injection, and then were challenged with a virulent CPV-2c isolate following a standard protocol at 3 weeks post-second vaccination.

Clinical observations: All animal were observed for fever (≧103.4° C.), clinical signs which include diarrhea, vomiting, mucous or blood in feces, lymphopenia (≧50% reduction prior to pre-challenge level) and fecal shedding. A puppy was considered as being positive for parvovirus disease if it was positive for 3 of the 4 pathogenomonic signs: lymphopenia, fever, fecal shedding, and clinical signs.

### Results and Conclusion

Prior to the challenge with CPV-2c, all puppies receiving the test vaccine were positive for CPV SN antibodies following the 2^{nd} vaccination dose (>4096), whereas all puppies receiving the placebo vaccine were free of CPV SN antibodies (<2). Following the CPV-2c challenge, 100% of the puppies that had been administered the placebo vaccine were positive for parvovirus disease, whereas 100% of the puppies vaccinated with the vaccine comprising the CPV-2c isolate #4 (La, the test vaccine) were free of parvovirus disease.

TABLE 3
EFFICACY OF TEST VACCINE WITH CPV-2c ISOLATE #
4 FOLLOWING A CHALLENGE WITH VIRULENT CPV-2c.

| Treatment | | | | % Dogs |
|---|---|---|---|---|
| Group | No. of dogs | Vaccine | Challenge virus | protected |
| 1 | 20 | Test Vaccine | CPV-2c | 100 |
| 2 | 5 | Placebo | CPV-2c | 0 |

The dogs were vaccinated subcutaneously with either two doses of the Test vaccine or two doses of the placebo prior to the challenge with CPV-2c

The results from this study (Table 3 above) demonstrate that the vaccine comprising the CPV-2c isolate #4 (ATCC accession No. PTA-13492) provided 100% protection against a virulent CPV-2c isolate. Indeed, although completely avirulent in dogs, the attenuated canine parvovirus of the present invention can still induce significant levels of protection against a CPV-2c challenge.

SEQUENCE LISTING TABLE

| SEQ ID NO: | Description |
|---|---|
| 1 | Nucleic acid sequence of VP2 |
| 2 | Amino acid sequence of VP2 |
| 3 | Nucleic acid sequence of VP1 |
| 4 | Amino acid sequence of VP1 |
| 5 | Nucleic acid sequence of NS2 |
| 6 | Amino acid sequence of NS2 |
| 7 | Nucleic acid sequence of NS1 |
| 8 | Amino acid sequence of NS1 |
| 9 | Nucleic acid sequence of >95% of the genome |

### Biological Deposit

A culture of the following biological material has been deposited with the following international depository by: Intervet Inc. 556 Morris Ave, Summit N.J., 07901.

American Type Culture Collection (ATCC) 10801 University Boulevard, Manassas, Va. 20110-2209, U.S.A., under conditions that satisfy the requirements of the Budapest Treaty on the International Recognition of the Deposit of Microorganisms for the Purposes of Patent Procedure.

| Organism | Accession No. | Date of Deposit |
|---|---|---|
| CPV-2c (isolate #4) | ATCC accession No. PTA-13492 | Jan. 24, 2013 |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It is further to be understood that all base sizes or amino acid sizes, and all molecular weight or molecular mass values, given for nucleic acids or polypeptides are approximate, and are provided for description.

1. An isolated attenuated canine parvovirus (CPV) isolate comprising a genome that encodes a capsid protein comprising an amino acid sequence that comprises 98% or greater identity with the amino acid sequence of SEQ ID NO: 2; wherein the amino acid sequence of the capsid protein comprises a glutamic acid residue at position 426 (E₄₂₆), and lysine residues at amino acid positions 93 (K₉₃), 219 (K₂₁₉), and 377 (K₃₇₇).

2. The isolated attenuated CPV of claim 1, wherein said capsid protein further comprises an amino acid residue selected from the group consisting of a serine residue at position 300 (S₃₀₀), an alanine residue at position 301 (A₃₀₁), and an isoleucine residue at position 555 (I₅₅₅), or any combination thereof.

3. The isolated attenuated CPV of claim 2, wherein the amino acid sequence of the capsid protein comprises a serine residue at position 300 (S₃₀₀).

4. The isolated attenuated CPV of claim 3, wherein the amino acid sequence of the capsid protein comprises an alanine residue at position 301 (A₃₀₁).

5. The isolated attenuated CPV of claim 2, wherein the amino acid sequence of the capsid protein comprises an isoleucine residue at position 555 (I₅₅₅).

6. An isolated attenuated canine parvovirus (CPV) isolate comprising the identifying characteristics of ATCC accession No. PTA-13492.

7. A vaccine comprising the attenuated CPV of claim 1 and a pharmaceutically acceptable carrier.

8. The vaccine of claim 7 further comprising an additional live attenuated canine virus selected from the group consisting of canine distemper virus, canine adenovirus type 2, canine parvovirus type 2b, canine parainfluenza virus, canine coronavirus, canine influenza virus, canine pneumovirus, or any combination thereof.

9. The vaccine of claim 8 that comprises a live attenuated canine distemper virus, a live attenuated canine adenovirus type 2, and a live attenuated canine parainfluenza virus.

10. The vaccine of claim 9 that further comprises a live attenuated canine coronavirus.

11. A method of immunizing a canine against CPV comprising administering the vaccine of claim 9 to a canine.

12. An immunogenic composition comprising the CPV of claim 1 and a pharmaceutically acceptable carrier.

13. A polypeptide that comprises an amino acid sequence that comprises 98% or greater identity with the amino acid sequence of SEQ ID NO: 2 or an antigenic fragment thereof;
wherein the amino acid sequence of the polypeptide or that of the antigenic fragment thereof comprises a glutamic acid residue at position 426 (E₄₂₆) and an amino acid residue selected from the group consisting of a lysine residue at position 93 (K₉₃), a lysine residue at position 219 (K₂₁₉), a lysine residue at position 377 (K₃₇₇), an isoleucine residue at position 555 (I₅₅₅), a serine residue at position 300 (S₃₀₀), an alanine residue at position 301 (A₃₀₁), or any combination thereof; and wherein said polypeptide or the antigenic fragment thereof is in a form selected from the group consisting of isolated, recombinant, or both isolated and recombinant.

14. The polypeptide of claim 13 that comprises lysine residues at amino acid positions 93 (K₉₃), 219 (K₂₁₉), and 377 (K₃₇₇).

15. The polypeptide of claim 14, wherein the amino acid sequence comprises a serine residue at position 300 (S₃₀₀), an alanine residue at position 301 (A₃₀₁), and an isoleucine residue at position 555 (I₅₅₅).

16. An immunogenic composition comprising the polypeptide or antigenic fragment thereof of claim 13.

17. A nucleic acid that encodes the polypeptide of claim 14; wherein said nucleic acid is in a form selected from the group consisting of isolated, recombinant, or both isolated and recombinant.

18. The nucleic acid of claim 17 that comprises the nucleotide sequence of SEQ ID NO: 1.

19. A recombinant expression vector that comprises the recombinant nucleic acid of claim 17.

20. The recombinant expression vector of claim 19 that is a recombinant viral vector.


SEQUENCE LISTING (9 sequences)

SEQ ID NO: 1 - DNA, 1755 bp, canine parvovirus
atgagtgatggagcagttcaaccagacggtggtcaacctgctgtcagaaatgaaagagctacaggatctgggaacgggtctggaggcgggggtggtggtggttctgggggtgtggggatttctacgggtacttttaataatcagacggaatttaaatttttggaaaacggatgggtggaaatcacagcaaactcaagcagacttgtacatttaaatatgccagaaagtgaaaattatagaagagtggttgtaaataatttggataaaactgcagttaaaggaaacatggctttagatgatactcatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaaaaccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttctaaattctttgcctcaagctgaaggaagtgctaactttggttatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacagatgaaaatcaagcagcagatggtgatccaaaatatgcatttggtagacaacatggtcaaaaaactaccacaacaggagaaacacctgagagatttacatatatagcacatcaagatacaggaagatatccagaaggagattggattcaaaatattaactttaaccttcctgtaacagaagataatgtattgctaccaacagatccaattggaggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatatagataaccaatttaactatgtaccaagtaatattggaggtatgaaaattgtatatgaaaaatctcaactagcacctagaaaattatattaa

SEQ ID NO: 2 - protein, 584 aa, canine parvovirus
MSDGAVQPDGGQPAVRNERATGSGNGSGGGGGGGSGGVGISTGTFNNQTEFKFLENGWVEITANSSRLVHLNMPESENYRRVVVNNLDKTAVKGNMALDDTHAQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLKPSHTGTSGTPTNIYHGTDPDDVQFYTIENSVPVHLLRTGDEFATGTFFFDCKPCRLTHTWQTNRALGLPPFLNSLPQAEGSANFGYIGVQQDKRRGVTQMGNTNYITEATIMRPAEVGYSAPYYSFEASTQGPFKTPIAAGRGGAQTDENQAADGDPKYAFGRQHGQKTTTTGETPERFTYIAHQDTGRYPEGDWIQNINFNLPVTEDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINIDNQFNYVPSNIGGMKIVYEKSQLAPRKLY

SEQ ID NO: 3 - DNA, 2184 bp, canine parvovirus
atggcacctccggcaaagagagccaggagaggacttgtgcctccaggttataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcaacctgctgtcagaaatgaaagagctacaggatctgggaacgggtctggaggcgggggtggtggtggttctgggggtgtggggatttctacgggtacttttaataatcagacggaatttaaatttttggaaaacggatgggtggaaatcacagcaaactcaagcagacttgtacatttaaatatgccagaaagtgaaaattatagaagagtggttgtaaataatttggataaaactgcagttaaaggaaacatggctttagatgatactcatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaaaaccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttctaaattctttgcctcaagctgaaggaagtgctaactttggttatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacagatgaaaatcaagcagcagatggtgatccaaaatatgcatttggtagacaacatggtcaaaaaactaccacaacaggagaaacacctgagagatttacatatatagcacatcaagatacaggaagatatccagaaggagattggattcaaaatattaactttaaccttcctgtaacagaagataatgtattgctaccaacagatccaattggaggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatatagataaccaatttaactatgtaccaagtaatattggaggtatgaaaattgtatatgaaaaatctcaactagcacctagaaaattatattaa

SEQ ID NO: 4 - protein, 727 aa, canine parvovirus
MAPPAKRARRGLVPPGYKYLGPGNSLDQGEPTNPSDAAAKEHDEAYAAYLRSGKNPYLYFSPADQRFIDQTKDAKDWGGKIGHYFFRAKKAIAPVLTDTPDHPSTSRPTKPTKRSKPPPHIFINLAKKKKAGAGQVKRDNLAPMSDGAVQPDGGQPAVRNERATGSGNGSGGGGGGGSGGVGISTGTFNNQTEFKFLENGWVEITANSSRLVHLNMPESENYRRVVVNNLDKTAVKGNMALDDTHAQIVTPWSLVDANAWGVWFNPGDWQLIVNTMSELHLVSFEQEIFNVVLKTVSESATQPPTKVYNNDLTASLMVALDSNNTMPFTPAAMRSETLGFYPWKPTIPTPWRYYFQWDRTLKPSHTGTSGTPTNIYHGTDPDDVQFYTIENSVPVHLLRTGDEFATGTFFFDCKPCRLTHTWQTNRALGLPPFLNSLPQAEGSANFGYIGVQQDKRRGVTQMGNTNYITEATIMRPAEVGYSAPYYSFEASTQGPFKTPIAAGRGGAQTDENQAADGDPKYAFGRQHGQKTTTTGETPERFTYIAHQDTGRYPEGDWIQNINFNLPVTEDNVLLPTDPIGGKTGINYTNIFNTYGPLTALNNVPPVYPNGQIWDKEFDTDLKPRLHVNAPFVCQNNCPGQLFVKVAPNLTNEYDPDASANMSRIVTYSDFWWKGKLVFKAKLRASHTWNPIQQMSINIDNQFNYVPSNIGGMKIVYEKSQLAPRKLY

SEQ ID NO: 5 - DNA, 498 bp, canine parvovirus
atgtctggcaaccagtatactgaggaagttatggagggagtaaattggttaaagaaacatgcagagaatgaagcattttcgtttgtttttaaatgtgacaacgtccaactaaatggaaaggatgttcgctggaacaactataccaaaccaattcaaaatgaagagctaacatctttaattagaggagcacaaacagcaatggatcaaaccgaagaagaagaaatggactgggaatcggaagttgatagtctcgccaaaaagttgcaaagacttagagacgcaagcggcaagcaatcctcagagtcaagaccaagctctaactcctctgactccgaacgtagtggaccttgcactggaaccgtggagtactccagatacgcctattgcaaaaactgcaaatcaacaatcaaaccaacttggcgttactcacaaagacgtgcaagcgagtccaacgtggtccgaaatagaggcagacctgagagccatctttacttctga

SEQ ID NO: 6 - protein, 165 aa, canine parvovirus
MSGNQYTEEVMEGVNWLKKHAENEAFSFVFKCDNVQLNGKDVRWNNYTKPIQNEELTSLIRGAQTAMDQTEEEEMDWESEVDSLAKKLQRLRDASGKQSSESRPSSNSSDSERSGPCTGTVEYSRYAYCKNCKSTIKPTWRYSQRRASESNVVRNRGRPESHLYF

SEQ ID NO: 7 - DNA, 2007 bp, canine parvovirus
atgtctggcaaccagtatactgaggaagttatggagggagtaaattggttaaagaaacatgcagagaatgaagcattttcgtttgtttttaaatgtgacaacgtccaactaaatggaaaggatgttcgctggaacaactataccaaaccaattcaaaatgaagagctaacatctttaattagaggagcacaaacagcaatggatcaaaccgaagaagaagaaatggactgggaatcggaagttgatagtctcgccaaaaagcaagtacaaacttttgatgcattaattaaaaaatgtctttttgaagtctttgtttctaaaaatatagaaccaaatgaatgtgtttggtttattcaacatgaatggggaaaagatcaaggctggcattgtcatgttttacttcatagtaagaacttacaacaagcaactggtaaatggctacgcagacaaatgaatatgtattggagtagatggttggtgactctttgttcggtaaatttaacaccaactgaaaagattaagctcagagaaattgcagaagatagtgaatgggtgactatattaacatacagacataagcaaacaaaaaaagactatgttaaaatggttcattttggaaatatgatagcatattactttttaacaaagaaaaaaattgtccacatgacaaaagaaagtggctattttttaagtactgattctggttggaaatttaactttatgaagtatcaagacagacaaattgtcagcacactttacactgaacaaatgaaaccagaaaccgttgaaaccacagtgacgacagcacaggaaacaaagcgcgggagaattcaaactaaaaaggaagtatcaatcaaatgtactttgcgggacttggttagtaaaagagtaacatcacctgaagactggatgatgttacaaccagatagttatattgaaatgatggcacaaccaggaggtgaaaatcttttaaaaaatacacttgaaatttgtactttgactttagcaagaacaaaaacagcatttgaattaatacttgaaaaagcagataatactaaactgactaactttgatcttgcaaattctagaacatgtcagatttttagaatgcacggatggaattggattaaagtttgtcacgctatagcatgtgttttaaatagacaaggtggtaaaagaaatacagttctttttcatggaccagcaagtacaggaaaatctatcattgctcaagccatagcacaagctgtgggtaatgttggttgttataatgcagcaaatgtaaattttccatttaatgactgcaccaataaaaatttaatttggattgaagaagctggtaactttggtcaacaagttaatcaatttaaagcaatttgttccggacaaacaattagaattgatcaaaaaggtaaaggaagtaagcaaattgaaccaactccagtaattatgacaactaatgaaaatataacaattgtgagaattggatgtgaagaaagacctgaacatacacaaccaataagagacagaatgttgaacattaagttagtatgtaagcttccaggagactttggtttggttgataaagaagaatggcctttaatatgtgcatggttagttaaacatggttatgaatcaaccatggctaactatacacatcattggggaaaagtaccagaatgggatgaaaactgggcggagcctaaaatacaagaaggtataaattcaccaggttgcaaagacttagagacgcaagcggcaagcaatcctcagagtcaagaccaagctctaactcctctgactccgaacgtagtggaccttgcactggaaccgtggagtactccagatacgcctattgcaaaaactgcaaatcaacaatcaaaccaacttggcgttactcacaaagacgtgcaagcgagtccaacgtggtccgaaatagaggcagacctgagagccatctttacttctgaacaactggaagaagattttcaagacgacttggattaa

SEQ ID NO: 8 - protein, 668 aa, canine parvovirus
MSGNQYTEEVMEGVNWLKKHAENEAFSFVFKCDNVQLNGKDVRWNNYTKPIQNEELTSLIRGAQTAMDQTEEEEMDWESEVDSLAKKQVQTFDALIKKCLFEVFVSKNIEPNECVWFIQHEWGKDQGWHCHVLLHSKNLQQATGKWLRRQMNMYWSRWLVTLCSVNLTPTEKIKLREIAEDSEWVTILTYRHKQTKKDYVKMVHFGNMIAYYFLTKKKIVHMTKESGYFLSTDSGWKFNFMKYQDRQIVSTLYTEQMKPETVETTVTTAQETKRGRIQTKKEVSIKCTLRDLVSKRVTSPEDWMMLQPDSYIEMMAQPGGENLLKNTLEICTLTLARTKTAFELILEKADNTKLTNFDLANSRTCQIFRMHGWNWIKVCHAIACVLNRQGGKRNTVLFHGPASTGKSIIAQAIAQAVGNVGCYNAANVNFPFNDCTNKNLIWIEEAGNFGQQVNQFKAICSGQTIRIDQKGKGSKQIEPTPVIMTTNENITIVRIGCEERPEHTQPIRDRMLNIKLVCKLPGDFGLVDKEEWPLICAWLVKHGYESTMANYTHHWGKVPEWDENWAEPKIQEGINSPGCKDLETQAASNPQSQDQALTPLTPNVVDLALEPWSTPDTPIAKTANQQSNQLGVTHKDVQASPTWSEIEADLRAIFTSEQLEEDFQDDLD

SEQ ID NO: 9 - DNA, 5058 bp, canine parvovirus
  misc_feature (4908)..(5058): n is a, c, g, or t
atcattctttagaaccaactgaccaagttcacgtacgtatgacgtgatgacgcgcgctgcgcgcgctgcctacggcagtcacacgtcatacgtacgctccttggtcagttggttctaaagaatgataggcggtttgtgtgtttaaacttgggcgggaaaaggtggcgggctaattgtgggcgtggttaaaggtataaaagacaaaccatagaccgttactgacattcgcttcttgtctttgacagagtgaacctctcttactttgactaaccatgtctggcaaccagtatactgaggaagttatggagggagtaaattggttaaagaaacatgcagagaatgaagcattttcgtttgtttttaaatgtgacaacgtccaactaaatggaaaggatgttcgctggaacaactataccaaaccaattcaaaatgaagagctaacatctttaattagaggagcacaaacagcaatggatcaaaccgaagaagaagaaatggactgggaatcggaagttgatagtctcgccaaaaagcaagtacaaacttttgatgcattaattaaaaaatgtctttttgaagtctttgtttctaaaaatatagaaccaaatgaatgtgtttggtttattcaacatgaatggggaaaagatcaaggctggcattgtcatgttttacttcatagtaagaacttacaacaagcaactggtaaatggctacgcagacaaatgaatatgtattggagtagatggttggtgactctttgttcggtaaatttaacaccaactgaaaagattaagctcagagaaattgcagaagatagtgaatgggtgactatattaacatacagacataagcaaacaaaaaaagactatgttaaaatggttcattttggaaatatgatagcatattactttttaacaaagaaaaaaattgtccacatgacaaaagaaagtggctattttttaagtactgattctggttggaaatttaactttatgaagtatcaagacagacaaattgtcagcacactttacactgaacaaatgaaaccagaaaccgttgaaaccacagtgacgacagcacaggaaacaaagcgcgggagaattcaaactaaaaaggaagtatcaatcaaatgtactttgcgggacttggttagtaaaagagtaacatcacctgaagactggatgatgttacaaccagatagttatattgaaatgatggcacaaccaggaggtgaaaatcttttaaaaaatacacttgaaatttgtactttgactttagcaagaacaaaaacagcatttgaattaatacttgaaaaagcagataatactaaactgactaactttgatcttgcaaattctagaacatgtcagatttttagaatgcacggatggaattggattaaagtttgtcacgctatagcatgtgttttaaatagacaaggtggtaaaagaaatacagttctttttcatggaccagcaagtacaggaaaatctatcattgctcaagccatagcacaagctgtgggtaatgttggttgttataatgcagcaaatgtaaattttccatttaatgactgcaccaataaaaatttaatttggattgaagaagctggtaactttggtcaacaagttaatcaatttaaagcaatttgttccggacaaacaattagaattgatcaaaaaggtaaaggaagtaagcaaattgaaccaactccagtaattatgacaactaatgaaaatataacaattgtgagaattggatgtgaagaaagacctgaacatacacaaccaataagagacagaatgttgaacattaagttagtatgtaagcttccaggagactttggtttggttgataaagaagaatggcctttaatatgtgcatggttagttaaacatggttatgaatcaaccatggctaactatacacatcattggggaaaagtaccagaatgggatgaaaactgggcggagcctaaaatacaagaaggtataaattcaccaggttgcaaagacttagagacgcaagcggcaagcaatcctcagagtcaagaccaagctctaactcctctgactccgaacgtagtggaccttgcactggaaccgtggagtactccagatacgcctattgcaaaaactgcaaatcaacaatcaaaccaacttggcgttactcacaaagacgtgcaagcgagtccaacgtggtccgaaatagaggcagacctgagagccatctttacttctgaacaactggaagaagattttcaagacgacttggattaaggtacgatggcacctccggcaaagagagccaggagaggtaagggtgtgttagtaaagtggggggaggggaaagatttagtaacttaactaagtatgtgtttttttataggacttgtgcctccaggttataaatatcttgggcctgggaacagtcttgaccaaggagaaccaactaacccttctgacgccgctgcaaaagaacacgacgaagcttacgctgcttatcttcgctctggtaaaaacccatacttatatttctcgccagcagatcaacgctttatagatcaaactaaggacgctaaagattggggggggaaaataggacattatttttttagagctaaaaaggcaattgctccagtattaactgatacaccagatcatccatcaacatcaagaccaacaaaaccaactaaaagaagtaaaccaccacctcatattttcatcaatcttgcaaaaaaaaaaaaagccggtgcaggacaagtaaaaagagacaatcttgcaccaatgagtgatggagcagttcaaccagacggtggtcaacctgctgtcagaaatgaaagagctacaggatctgggaacgggtctggaggcgggggtggtggtggttctgggggtgtggggatttctacgggtacttttaataatcagacggaatttaaatttttggaaaacggatgggtggaaatcacagcaaactcaagcagacttgtacatttaaatatgccagaaagtgaaaattatagaagagtggttgtaaataatttggataaaactgcagttaaaggaaacatggctttagatgatactcatgcacaaattgtaacaccttggtcattggttgatgcaaatgcttggggagtttggtttaatccaggagattggcaactaattgttaatactatgagtgagttgcatttagttagttttgaacaagaaatttttaatgttgttttaaagactgtttcagaatctgctactcagccaccaactaaagtttataataatgatttaactgcatcattgatggttgcattagatagtaataatactatgccatttactccagcagctatgagatctgagacattgggtttttatccatggaaaccaaccataccaactccatggagatattattttcaatgggatagaacattaaaaccatctcatactggaactagtggcacaccaacaaatatataccatggtacagatccagatgatgttcaattttatactattgaaaattctgtgccagtacacttactaagaacaggtgatgaatttgctacaggaacatttttttttgattgtaaaccatgtagactaacacatacatggcaaacaaatagagcattgggcttaccaccatttctaaattctttgcctcaagctgaaggaagtgctaactttggttatataggagttcaacaagataaaagacgtggtgtaactcaaatgggaaatacaaactatattactgaagctactattatgagaccagctgaggttggttatagtgcaccatattattcttttgaggcgtctacacaagggccatttaaaacacctattgcagcaggacgggggggagcgcaaacagatgaaaatcaagcagcagatggtgatccaaaatatgcatttggtagacaacatggtcaaaaaactaccacaacaggagaaacacctgagagatttacatatatagcacatcaagatacaggaagatatccagaaggagattggattcaaaatattaactttaaccttcctgtaacagaagataatgtattgctaccaacagatccaattggaggtaaaacaggaattaactatactaatatatttaatacttatggtcctttaactgcattaaataatgtaccaccagtttatccaaatggtcaaatttgggataaagaatttgatactgacttaaaaccaagacttcatgtaaatgcaccatttgtttgtcaaaataattgtcctggtcaattatttgtaaaagttgcgcctaatttaacaaatgaatatgatcctgatgcatctgctaatatgtcaagaattgtaacttactcagatttttggtggaaaggtaaattagtatttaaagctaaactaagagcctctcatacttggaatccaattcaacaaatgagtattaatatagataaccaatttaactatgtaccaagtaatattggaggtatgaaaattgtatatgaaaaatctcaactagcacctagaaaattatattaacatacttactatgtttttatgtttattacatattattttaagattaattaaattacagcatagaaatattgtacttgtacttgatataggatttagaaggtttgttatatggtatacaataactgtaagaaatagaagaacatttagatcatagttagtagtttgttttgtaaaatgtattgtaaaccattaatgtatgttgttatggtgtgggtggttggttggtttgcccttagaatatgttaaggaccaaaaaaaatcaataaaagacatttaaaactaaatggcctcgtatactgtctataaggtgaactaaccttaccataagtatcaatctgtctttaaggggggggtgggtgggagatgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn